(19) United States
(12) Patent Application Publication
(10) Pub. No.: US 2014/0371098 A1
(43) Pub. Date: Dec. 18, 2014

(54) MARKER SEQUENCES FOR BREAST CANCER AND THE USE THEREOF

(71) Applicant: Protagen AG, Dortmund (DE)

(72) Inventors: Angelika Lüking, Bochum (DE); Axel Kowald, Bochum (DE); Annabel Höpner, Bielefeld (DE); Christian Scheer, Dortmund (DE); Heidelinde Fiegl, Ellbögen (AT); Günter Daxenbichler, Innsbruck (AT)

(73) Assignee: Protagen AG, Dortmund (DE)

(21) Appl. No.: 14/367,276

(22) PCT Filed: Dec. 24, 2012
(86) PCT No.: PCT/EP2012/076875

§ 371 Date: Aug. 25, 2014

(30) Foreign Application Priority Data
Dec. 22, 2011 (EP) 11195459.0

(51) Int. Cl. G01N 33/574

(52) U.S. Cl. CPC G01N 33/57415; G01N 2500/04; G01N 2500/20
USPC 506/9; 530/350; 506/18; 536/23.5

(57) ABSTRACT

The present invention relates to a method for identifying marker sequences for breast cancer, the marker sequences identified with the aid of this method and diagnostic use thereof, diagnostic devices containing marker sequences for breast cancer, in particular an arrangement and a protein array, and use thereof. The invention also relates to method for the screening of potential active agents for the treatment and prevention of breast cancer by means of these marker sequences.

The present invention relates to a method for identifying marker sequences for breast cancer, the marker sequences identified with the aid of this method and diagnostic use thereof, diagnostic devices containing marker sequences for breast cancer, in particular an arrangement and a protein array, and use thereof. The invention also relates to methods for screening potential active agents for the treatment and prevention of breast cancer by means of these marker sequences.

Protein arrays are gaining increasing industrial importance in analysis and diagnosis as well as in pharmaceutical development. Protein arrays have become established as screening tools.

Here, the rapid and highly parallel detection of a multiplicity of specifically binding analysis molecules in a single experiment is made possible. To produce protein arrays, it is necessary to have the required proteins available. In particular, protein expression libraries have been established for this purpose. High-throughput cloning of defined open reading frames is one possibility (Heyman, J. A., Cornthwaite, J., Foncerrada, L., Gilmore, J. R., Gontang, E., Hartman, K. J., Hernandez, C. L., Hood, R., Hull, H. M., Lee, W. Y., Marcil, R., Marsh, E. J., Mudd, K. M., Patino, M. J., Purcell, T. J., Rowland, J. J., Sindici, M. L. and Hoeffler, J. P. (1999) Genome-scale cloning and expression of individual open reading frames using topoisomerase I-mediated ligation. Genome Res, 9, 383-392; Kersten, B., Feilner, T., Kramer, A., Wehrmeyer, S., Possling, A., Witt, I., Zanor, M. I., Stracke, R., Lueking, A., Kreutzberger, J., Lehrach, H. and Cahill, D. J. (2003) Generation of Arabidopsis protein chip for antibody and serum screening. Plant Molecular Biology, 52, 999-1010; Reboul, J., Vaglio, P., Rual, J. F., Lamesch, P., Martinez, M., Armstrong, C. M., Li, S., Jacotot, L., Bertin, N., Janky, R., Moore, T., Hudson, J. R., Jr., Hartley, J. L., Brasch, M. A., Vandenhaute, J., Boulton, S., Endress, G. A., Jenna, S., Chevet, E., Papasotiropoulos, V., Tolias, P. P., Ptacek, J., Snyder, M., Huang, R., Chance, M. R., Lee, H., Doucette-Stamm, L., Hill, D. E. and Vidal, M. (2003) C. elegans ORFeome version 1.1: experimental verification of the genome annotation and resource for proteome-scale protein expression. Nat Genet, 34, 35-41; Walhout, A. J., Temple, G. F., Brasch, M. A., Hartley, J. L., Lorson, M. A., van den Heuvel, S. and Vidal, M. (2000) GATEWAY recombinational cloning: application to the cloning of large numbers of open reading frames or ORFeomes. Methods Enzymol, 328, 575-592). However, such an approach is closely linked to the progress of the genome sequencing projects and the annotation of these gene sequences. In addition, the determination of the expressed sequence is not always clear due to differential splicing processes.

This problem can be avoided by the use of cDNA expression libraries (Bussow, K., Cahill, D., Nietfeld, W., Bancroft, D., Scherzinger, E., Lehrach, H. and Walter, G. (1998) A method for global protein expression and antibody screening on high-density filters of an arrayed cDNA library. Nucleic Acids Research, 26, 5007-5008; Büssow, K., Nordhoff, E., Lübbert, C., Lehrach, H. and Walter, G. (2000) A human cDNA library for high-throughput protein expression screening. Genomics, 65, 1-8; Holz, C., Lueking, A., Bovekamp, L., Gutjahr, C., Bolotina, N., Lehrach, H. and Cahill, D. J. (2001) A human cDNA expression library in yeast enriched for open reading frames. Genome Res, 11, 1730-1735; Lueking, A., Holz, C., Gotthold, C., Lehrach, H. and Cahill, D. (2000) A system for dual protein expression in Pichia pastoris and Escherichia coli, Protein Expr. Purif., 20, 372-378). Here, the cDNA of a specific tissue is cloned into a bacterial or eukaryotic expression vector, such as yeast. The vectors used for the expression are generally characterised in that they carry inducible promoters that may be used to control the time of protein expression. In addition, expression vectors have sequences for what are known as affinity epitopes or affinity proteins, which on the one hand permit the specific detection of the recombinant fusion proteins by means of an antibody directed against the affinity epitope, and on the other hand the specific purification via affinity chromatography (IMAC) is rendered possible.

By way of example, the gene products of a cDNA expression library from human foetal brain tissue in the bacterial expression system Escherichia coli were arranged in high-density format on a membrane and could be successfully screened with different antibodies. It was possible to show that the proportion of full-length proteins is at least 66%. Additionally, the recombinant proteins from expression libraries could be expressed and purified in a high-throughput manner (Braun P., Hu, Y., Shen, B., Halleck, A., Koundinya, M., Harlow, E. and LaBaer, J. (2002) Proteome-scale purification of human proteins from bacteria. Proc Natl Acad Sci USA, 99, 2654-2659; Bussow (2000) supra; Lueking, A., Horn, M., Eickhoff, H., Bussow, K., Lehrach, H. and Walter, G. (1999) Protein microarrays for gene expression and antibody screening. Analytical Biochemistry, 270, 103-111). Such protein arrays based on cDNA expression libraries are disclosed in particular in WO 99/57311 and WO 99/57312.

Furthermore, in addition to antigen-presenting protein arrays, antibody-presenting arrangements are likewise described (Lal et al (2002) Antibody arrays: An embryonic but rapidly growing technology, DDT, 7, 143-149; Kusnezow et al. (2003), Antibody microarrays: An evaluation of production parameters, Proteomics, 3, 254-264).

Breast cancer (breast carcinoma) is the most common malignant tumour of the human mammary gland. It occurs primarily in women; only approximately every hundredth occurrence of these cancer diseases occurs in men. In Western countries, breast cancer is the most common form of cancer in women and more women die from breast cancer than from any other cancer disease. Most diseases occur sporadically (randomly), however there are hereditary and acquired risk factors. Numerous national and international programmes for the early detection and structured treatment are intended to reduce the mortality rate.

Approximately 80 to 90% of all tumours in the female breast are discovered randomly by the women themselves. These palpable and visible tumours are often relatively large when they are discovered and are therefore usually associated with a poor prognosis. By consistent early detection of smaller, impalpable tumours, the mortality rate could be reduced significantly. Programmes for systematic self-examination, palpation examination by the doctor and screening mammography with the aid of imaging methods as well as biopsy are used for early detection.

In the case of breast cancer, early diagnosis is key for the further development of the disease and the prognosis. Although numerous tumour markers are associated with breast cancer, these are not suitable for early detection, since they have inadequate specificity. They are used predominantly for the monitoring of the course of the disease or of the response to therapy.

US 2003/198972 A1 discloses the identification and use of gene expression patterns that are associated with different stages of breast cancer. The expression patterns are obtained here by comparative analysis of the gene expression in healthy female patients and in female patients with benign changes and in female breast cancer patients. In these studies, the sample is removed from the patients by non-invasive flushing of the mammary duct, is examined microscopically and degenerated cells are harvested from the sample. RNA is extracted from these cells, amplified, labelled and then brought into contact with microarrays equipped with polynucleotides. The intensity pattern is analysed and enables an assignment to different stages of breast cancer. US 2003/198972 A1 in this regard specifies individual genes of which the expression pattern is modified in conjunction with breast cancer. US 2003/198972, however, does not disclose any new marker sequences for detection of these genes.

Karen S. Anderson et al. (Journal of Proteom Research (2011) vol. 10, Nr. 1, pages 85-96) discloses the detection of autoantibodies against tumour-associated proteins with the aid of protein microarrays and the application for early detection of invasive breast cancer. The protein microarrays used here are produced in that full-length clones of the cDNAs coding for potential tumour-associated antigens are printed onto the support, expressed and then tested comparatively with the sera of female breast cancer patients and control individuals, specifically healthy subjects and those with benign breast diseases.

US 2012/021887 discloses the use of arrangements and protein microarrays with marker sequences for breast cancer for the detection of breast cancer-specific autoantibodies.

There is a need, however, for indication-specific diagnostic devices and methods for breast cancer, in particular for the early detection of breast cancer, for the distinction of breast cancer and benign changes to the breast and for the prediction of the risk of metastasis formation. The object of the present invention is to provide improved means for the early detection and therapy control in the case of breast cancer.

The invention relates to a method for identifying marker sequences for breast cancer, characterised in that
a. marker sequence candidates for breast cancer are identified in that a support, on which at least 1,000 different proteins are immobilised, is brought into contact with a serum sample from a female patient with breast cancer and proteins are identified that demonstrate an interaction with the serum (marker sequence candidates), and
b. the interaction of one or more marker sequence candidates from a. with the serum of female patients with breast cancer is determined compared with the interaction of the marker sequence candidate(s) from a. with the serum of female patients with benign changes and
   the interaction of the marker sequence candidate(s) from a. with the serum of healthy control individuals, and
c. marker sequences are identified in that they demonstrate an interaction with the serum from female patients with breast cancer that is different compared with the interaction with the serum from female patients with benign changes and the serum of healthy control individuals.

In one embodiment of the method, at least 5,000, preferably at least 10,000, different proteins are immobilised simultaneously on the support according to a.

For example, the comparative evaluation of the data concerning the interaction from b. is performed by means of statistical analysis, for example as described in the examples.

In a particularly preferred embodiment of the method, marker sequences for breast cancer are identified that are specific for breast cancer with a high risk of metastasis formation.

With the aid of the method according to the invention, marker sequences for breast cancer can be identified that are highly specific. Marker sequences that are found with this method on the one hand enable the early detection of breast cancer, for example of preliminary stages thereof, and on the other hand enable the distinction of breast cancer or preliminary stages thereof from benign changes. An early diagnosis and optionally a targeted treatment and also a considerably improved prognosis are thus possible. In addition, further patient data is included in the evaluation, such as a certain case history, lifestyle and in particular the risk of metastasis formation. Marker sequences for breast cancer that already in the early stage enable a prognosis with regard to the progression and/or the risk of metastasis formation can thus be identified with the method according to the invention. The prognosis with regard to the risk of metastasis formation also enables an improved therapy and a selective monitoring of the patient in view of metastatisation.

In contrast to the experiments described in the prior art by Karen S. Anderson et al. (above), marker sequences that are more specific, for example because they not only enable the discrimination of metastatisation from non-malignant changes of the tissue (benign change), but also enable an assertion with respect to the prognosis, for example in view of the risk of metastatisation, and thus the selective monitoring and therapy of these patients, are identified with the aid of the method according to the invention. The marker sequences according to the invention are particularly suitable for the analysis of bodily fluids, such as serum. A quick and cost-effective use or application of the marker sequences according to the invention is thus possible.

The invention also relates to the marker sequences for breast cancer identified with the method according to the invention. The invention relates to marker sequences for breast cancer obtainable by a method according to the invention and selected from the sequences comprising SEQ ID No. 1-1473 and partial sequences of SEQ ID No. 1-1473 with at least 90%, preferably 95%, of the length of the sequences SEQ ID No. 1-1473, and homologues of SEQ ID No. 1-1473 and partial sequences thereof with an identity of at least 95%, preferably 98% or more, to the corresponding nucleic acid and/or protein sequences, and sequences coded by SEQ ID No. 1-491, partial sequences thereof and homologues.

The invention also relates to an arrangement comprising one or more marker sequences according to the invention.

The invention also relates to a protein array comprising one or more marker sequences according to the invention.

The invention also relates to a diagnostic tool comprising one or more marker sequences according to the invention and optionally further additives and/or excipients.

The invention also relates to a test kit comprising one or more marker sequences according to the invention and optionally further additives and/or excipients.

The invention also relates to an arrangement according to the invention, characterised in that 2 or 3, preferably 4 or 5, particularly preferably 7 or 8 or more, different marker sequences for breast cancer are used simultaneously.

The invention also relates to a protein array according to the invention, characterised in that 2 or 3, preferably 4 or 5, particularly preferably 7 or 8 or more, different marker sequences for breast cancer are used simultaneously.

The invention also relates to a diagnostic tool according to the invention, characterised in that 2 or 3, preferably 4 or 5, particularly preferably 7 or 8 or more, different marker sequences for breast cancer are used simultaneously.

The invention also relates to a test kit according to the invention, characterised in that 2 or 3, preferably 4 or 5, particularly preferably 7 or 8 or more, different marker sequences for breast cancer are used simultaneously.

The invention also relates to the use of one or more marker sequences according to the invention, an arrangement according to the invention, a protein array according to the invention, a diagnostic tool according to the invention or a test kit according to the invention for the early detection, diagnosis, prognosis, therapy control and/or aftercare in the case of breast cancer.

The invention also relates to the use of one or more marker sequences according to the invention, an arrangement according to the invention, a protein array according to the invention, a diagnostic tool according to the invention or a test kit according to the invention for distinguishing breast cancer from benign changes and/or for prognosis, for example in respect of the risk of metastasis formation.

The invention also relates to the use of one or more marker sequences according to the invention, an arrangement according to the invention, a protein array according to the invention, a diagnostic tool according to the invention or a test kit according to the invention for individualised diagnosis and/or therapy in individual patients, patient groups, cohorts, population groups, variants of breast cancer, or stages of breast cancer.

The invention also relates to the use of one or more marker sequences according to the invention, an arrangement according to the invention, a protein array according to the invention, a diagnostic tool according to the invention or a test kit according to the invention for the detection and/or for the determination of the quantity of one or more breast cancer-associated autoantibodies, for example in bodily fluids, such as serum, tissue or tissue samples from the patient.

The invention also relates to the use of one or more marker sequences according to the invention, an arrangement according to the invention, a protein array according to the invention, a diagnostic tool according to the invention or a test kit according to the invention for the analysis of autoantibody profiles of patients, in particular for the qualitative and/or quantitative analysis of autoantibodies and/or for the monitoring of changes of autoantibody profiles, for example in bodily fluids such as serum, tissue or tissue samples from the patient.

The invention also relates to the use of one or more marker sequences according to the invention, an arrangement according to the invention, a protein array according to the invention, a diagnostic tool according to the invention or a test kit according to the invention for the screening of substances (active agents) for breast cancer.

The invention also relates to a target for the treatment and/or therapy of breast cancer, wherein the target is selected from the marker sequences SEQ ID No. 1-1473 according to the invention and partial sequences of SEQ ID No. 1-1473 with at least 90%, preferably 95%, of the length of sequences SEQ ID No. 1-1473, and homologues of SEQ ID No. 1-1473 and partial sequences thereof with an identity of at least 95%, preferably 98% or more, to the corresponding nucleic acid and/or protein sequences and sequences coded by SEQ ID No. 1-1491, partial sequences thereof and homologues thereof.

The invention also relates to a method for the early detection, diagnosis, prognosis, therapy control and/or aftercare in the case of breast cancer, wherein

a.) a marker sequence or a number of marker sequences selected from the group comprising the sequences SEQ ID No. 1-1473 and partial sequences of SEQ ID No. 1-1473 with at least 90%, preferably 95%, of the length of the sequences SEQ ID No. 1-1473, and homologues of SEQ ID No. 1-1473 and partial sequences thereof with an identity of at least 95%, preferably 98% or more, to the corresponding nucleic acid and/or protein sequences and sequences coded by SEQ ID No. 1-491, partial sequences thereof and homologues thereof is/are applied to a support,
b.) is/are brought into contact with bodily fluid or tissue sample from a patient, and
c.) an interaction of the bodily fluid or of the tissue sample with the marker sequence(s) for breast cancer from a.) is detected.

The breast cancer-specific marker sequences SEQ ID No. 1-491 have been described here for the first time. What is common to all of these sequences is that they have been identified by means of a protein array and the method described in the examples. The invention therefore also relates in particular to the breast cancer-specific marker sequence selected from the sequences comprising SEQ ID No. 1-491 and partial sequences of SEQ ID No. 1-491 with at least 90%, preferably 95%, of the length of the sequences SEQ ID No. 1-491, and homologues of SEQ ID No. 1-491 and partial sequences thereof with an identity of at least 95%, preferably at least 98% or more, to the corresponding sequences and proteins/peptides coded by the sequences SEQ ID No. 1-491, coded by the partial sequences, and the homologues.

Within the scope of this invention, a breast cancer-specific use for the proteins SEQ ID No. 983 to 1473 and/or partial sequences of these proteins and/or proteins coded by sequences SEQ ID No. 1-491 and/or proteins coded by sequences SEQ ID. No. 492-982 and/or coded by partial sequences of SEQ ID. No. 1-982 has been found for the first time and has been implemented in the arrangement according to the invention, the diagnostic tool according to the invention, the test kit according to the invention and the protein array according to the invention.

The invention thus provides marker sequences and arrangements of marker sequences for breast cancer that can be used within the scope of individualised diagnosis and therapy, for example in order to diagnose breast cancer and to monitor the therapy in a targeted and individually adapted manner in different patients, patient groups, cohorts, population groups, variants of breast cancer and stages of breast cancer.

The invention relates to the use of one or more marker sequences for breast cancer (=breast cancer-specific marker sequences), wherein the breast cancer-specific marker sequence(s) is/are selected from the sequences SEQ ID No. 1-1473 and partial sequences of SEQ ID No. 1-1473 with at least 90%, preferably at least 95%, of the length of SEQ ID No. 1-1473, and homologues of SEQ ID No. 1-1473 and partial sequences thereof with an identity of at least 95%, preferably at least 98% or more, to the corresponding sequences and proteins/peptides coded by the sequences SEQ ID No. 1-491, coded by partial sequences thereof, and homologues for breast cancer diagnosis and therapy, in particular for the early detection of breast cancer, for the diagnosis of breast cancer, for the prognosis, for example of the risk of metastasis formation, therapy control, for example prediction and monitoring of the response to a drug or a therapy, or aftercare. In particular, the invention also relates to the detection and the determination of the quantity of at least two different autoantibodies in a patient, wherein at least two different breast cancer-specific marker sequences according to the invention are used accordingly (as antigens).

The invention also relates to an arrangement according to the invention of one or more breast cancer-specific marker sequences on a support for the analysis of breast cancer-associated autoantibody profiles, early detection, diagnosis, prognosis and/or therapy control in the case of breast cancer, wherein the breast cancer-specific marker sequence(s) is/are selected from group SEQ ID No. 1-1473 and partial sequences of SEQ ID No. 1-1473 with at least 90%, preferably at least 95%, of the length of SEQ ID No. 1-1473, and homologues of SEQ ID No. 1-1473 and partial sequences thereof with an identity of at least 95%, preferably at least 98% or more, to the corresponding sequences and proteins/peptides coded by the sequences SEQ ID No. 1-491, coded by partial sequences thereof and homologues.

The invention also relates to an arrangement according to the invention or a use according to the invention of one or more breast cancer-specific marker sequences, wherein at least 2, for example 3 to 5 or 10, preferably 30 to 50 or 50 to 100 or more breast cancer-specific marker sequences are determined on or relative to a patient to be tested.

The invention also relates to a use, arrangement, protein array, diagnostic tool or test kit according to the invention, wherein the breast cancer-specific marker sequence(s) is/are applied to a solid support, in particular a filter, a membrane, a bead or small plate or bead, for example a magnetic or fluorophore-labelled bead, a silicon wafer, glass, metal, plastic, a chip, a mass spectrometry target or a matrix.

A particular embodiment concerns the use of a filter as a solid support. Furthermore, PVDF, nitrocellulose or nylon is preferred as a filter (for example Immobilon P Millipore, Protran Whatman, Hybond N+Amersham).

A further embodiment concerns an arrangement/use, characterised in that the breast cancer-specific marker sequence(s) is/are present as clone(s). The invention therefore relates to the use of breast cancer-specific marker sequences for the diagnosis of breast cancer, wherein at least one breast cancer-specific marker sequence of a DNA, in particular a cDNA selected from the group SEQ ID No. 1-491 or RNA selected from the group 492-982 or a partial sequence or a fragment or a homologue sequence thereof is determined on or relative to patients to be tested.

The provision of breast cancer-specific marker sequences (also referred to as marker sequences according to the invention) allows a reliable diagnosis and stratification of patients with breast cancer, in particular by means of a protein array.

The breast cancer-specific marker sequences according to the invention were able to be identified by means of differential screening of samples, more specifically from healthy test subjects, with patient samples with breast cancer. Here, these marker sequences according to the invention were able to be identified for the first time by means of protein arrays (see the examples).

The invention also relates to a method for identifying marker sequences for breast cancer, comprising the following steps:

a) providing sequences on an array,
b) identifying marker sequence candidates for breast cancer by comparative analysis of the signals measured in the event of contact of the marker sequences with bodily fluid or tissue sample from a patient with breast cancer and bodily fluid or tissue sample from a patient without breast cancer,
c) characterisation the marker sequence candidates for breast cancer with the aid of a protein array,
d) selecting marker sequences for breast cancer, which deliver a different signal in the case of patients with breast cancer and patients without breast cancer (breast cancer-specific marker sequences).

The term “breast cancer” comprises a group of diseases that can be preliminary stages of breast cancer and the establishment thereof as breast cancer or breast carcinoma (definition for example in accordance with Pschyrembel, de Gruyter, 263^{rd} edition (2012), Berlin). Variants of breast cancer and stages of breast cancer can also be inferred from the definition according to Pschyrembel.

In a further embodiment of the invention, the marker sequences according to the invention can also be combined with, supplemented or extended by known biomarkers for this indication. However, at least 50%, preferably 60%, particularly preferably 70% or more, marker sequences according to the invention are represented here, for example in the arrangement according to the invention, the protein array according to the invention, the diagnostic tool according to the invention or the tool kit according to the invention. In particularly preferred embodiments of the invention, in particular of the arrangement according to the invention, the assay according to the invention and protein array and also the use according to the invention, at least 75%, preferably 80% or 85%, particularly preferably 90% or 95%, of marker sequences according to the invention are represented.

In a preferred embodiment, the breast cancer-specific marker sequences are determined outside the human body, and the determination is performed in an ex vivo/in vitro diagnosis.

The invention also relates to an assay or protein array comprising an arrangement/use according to the invention. The invention relates to a diagnostic device and/or an assay, in particular a protein array, that allows an early detection, diagnosis, prognosis, stratification and/or testing for breast cancer.

The invention also relates to the use of an arrangement according to the invention or of an assay or protein array according to the invention for the analysis of autoantibody profiles of patients, in particular for the quantitative analysis and/or for the monitoring of changes of autoantibody profiles of patients.

The invention also relates to a diagnostic tool (test kit) for the early detection and/or diagnosis of breast cancer and/or prognosis and/or prediction of the risk of metastasis formation in the case of breast cancer, comprising an arrangement according to the invention, preferably on a support or an assay or protein array according to the invention and optionally further additives and excipients.

The invention also relates to a diagnostic tool (test kit) for therapy monitoring and/or aftercare in the case of breast cancer, comprising an arrangement according to the invention or an assay or protein array according to the invention and optionally further additives and excipients.

In a further embodiment of the invention, the invention relates to the use of breast cancer-specific marker sequences as diagnostic agents, wherein at least one marker sequence of a cDNA is selected from the group SEQ ID No. 1-491 (clone sequences) or SEQ ID No. 492-982 (RNA) or is a protein coded by SEQ ID No. 1-982 or a partial sequence or fragment thereof.

The invention also relates to a method for the early detection and diagnosis of breast cancer, wherein

a.) a breast cancer-specific marker sequence or a number of breast cancer-specific marker sequences selected from the group of sequences SEQ ID No. 1-1473 and partial sequences of SEQ ID No. 1-1473 with at least 90%, preferably at least 95%, of the length of SEQ ID No. 1-1473 and homologues of SEQ ID No. 1-1473 and partial sequences thereof with an identity of at least 95%, preferably at least 98%, or more, to the corresponding sequences and proteins/peptides coded by the sequences SEQ ID No. 1-491, coded by partial sequences thereof and homologies is/are applied to a support and
b.) is/are brought into contact with bodily fluid or tissue sample from a patient, and
c.) an interaction of the bodily fluid or tissue sample with the breast cancer-specific marker sequences from a.) is detected.

A particular embodiment of the invention concerns methods for the early detection and diagnosis of breast cancer, wherein the interaction according to c.) indicates a breast cancer-associated autoantibody profile of the patient or of a cohort or of a population group or of a specific disease progression (prognosis) or of a certain response to a therapy/drug.

A breast cancer-specific marker sequence or a number of breast cancer-specific marker sequences is/are used in a diagnosis method and/or in a diagnostic agent and/or in a test kit. In a preferred embodiment, at least 2, for example 3, 4, 5, 6, 7, 8, 9, 10, preferably 15 to 20 marker sequences or 30 to 50 or 100 or more breast cancer-specific marker sequences are used together or in combination, for example directly in succession or in parallel.

An interaction of the bodily fluid or of the tissue sample with the breast cancer-specific marker sequence or marker sequences can be detected for example by means of a probe, in particular by means of an antibody.

A particular embodiment of the invention relates to the method, wherein the stratification or the therapy control comprises decisions for the treatment and therapy of the patient, in particular hospitalisation of the patient, use, efficacy and/or dosage of one or more drugs, a therapeutic measure or the monitoring of the course of a disease and the course of therapy, aetiology or classification of a disease including prognosis. The invention also relates to a method for the stratification, in particular the risk stratification and/or therapy control of a patient with breast cancer.

The stratification of patients with breast cancer in new or established sub-groups of patients with breast cancer, and the appropriate selection of patient groups for the clinical development of new therapeutic agents is also included. The term “therapy control” also includes the allocation of patients to responders and non-responders regarding a therapy or the therapy course thereof.

In the sense of this invention, “diagnosis” means the positive determination of breast cancer by means of the breast cancer-specific marker sequences according to the invention as well as the assignment of the patients to breast cancer. The term diagnosis includes the medical diagnostics and examinations in this regard, in particular in-vitro diagnostics and laboratory diagnostics, and also proteomics and nucleic acid blotting. Further tests may be necessary to be sure and to exclude other diseases. The term diagnosis therefore also includes the differential diagnosis of breast cancer by means of the breast cancer-specific marker sequences according to the invention, and the prognosis of breast cancer, in particular the prediction of the risk of metastasis formation.

In the sense of this invention, “stratification or therapy control” means that, for example, the methods according to the invention allow decisions for the treatment and therapy of the patient, whether it is the hospitalisation of the patient, the use, efficacy and/or dosage of one or more drugs, a therapeutic measure or the monitoring of the course of a disease and the course of therapy or aetiology or classification of a disease, for example into a new or existing sub-type, or the differentiation of diseases and patients thereof.

In a further embodiment of the invention, the term “stratification” in particular includes the risk stratification with the prognosis of an “outcome” of a negative health event.

“Prognosis” means the prediction of the course of a disease, for example the prediction of the relapse-free survival, the overall survival, or the risk of metastasis formation.

Within the scope of this invention, the term “patient” is understood to mean any test subject (human or mammal), with the provision that the test subject is tested for breast cancer.

The term “breast cancer-specific marker sequence(s)” in the context of this invention means that that the nucleic acid, for example DNA, in particular cDNA or RNA or the coded amino acid sequence or the polypeptide or protein obtainable therefrom are significant (specific) for breast cancer. Breast cancer-specific marker sequences can be nucleic acid sequences and amino acid sequences, wherein modifications are also included.

The expressions “breast cancer-specific” and “for breast cancer” mean that, for example, the cDNA or the polypeptide or protein obtainable therefrom interacts with substances from the bodily fluid or tissue sample from a patient with breast cancer (for example antigen (epitope)/antibody (paratope) interaction). These substances from the bodily fluid or tissue sample either only occur or are only expressed, or occur or are expressed at least in an intensified manner, in the case of breast cancer, whereas these substances in patients or individuals without breast cancer are not present or are only present to a smaller extent (smaller quantity, lower concentration). On the other hand, breast cancer-specific marker sequences can also be characterised in that they interact with substances from the bodily fluid or tissue sample from patients with breast cancer because these substances no longer occur or are no longer expressed, or occur or are expressed at least in a much lower quantity/concentration, in the case of breast cancer, whereas these substances are present or are present at least to a much greater extent in patients or individuals without breast cancer. Breast cancer-specific marker sequences (marker sequences for breast cancer) may also be present in healthy test subjects, however the quantity (concentration) thereof changes for example with the development, establishment and therapy of breast cancer. The breast cancer-specific marker sequences are therefore biomarkers for breast cancer. The breast cancer-specific marker sequences may thus indicate a profile of substances from bodily fluid and tissue sampling, for example a breast cancer-associated autoantibody profile.

Autoantibody profiles comprise the quantity of one or more autoantibodies of which the occurrence/expression accompanies the development and/or establishment of breast cancer. “Breast cancer-associated autoantibody profiles” thus include on the one hand the composition (one or more autoantibodies) and on the other the quantity/concentration of individual autoantibodies.

In a particularly preferred embodiment of the invention, the breast cancer-specific marker sequence is an antigen or part of an antigen or codes for an antigen or for part of an antigen.

In a particularly preferred embodiment, the breast cancer-specific marker sequence identifies/binds to autoantibodies that are present (intensified) during the course of the development, establishment and therapy of breast cancer or are present to a smaller extent (or are no longer present) (referred to hereinafter as “breast cancer-associated autoantibodies”). Autoantibodies are formed by the body against the body's own antigens, which for example are produced when breast cancer is present. Autoantibodies are formed by the body against different substances and pathogens. Within the scope of the present invention, the breast cancer-associated autoantibodies in particular that are formed with the occurrence of and during the course of the development of breast cancer and/or of which the expression is upregulated or downregulated are detected. Breast cancer-associated autoantibodies can be detected with the aid of the method according to the invention and breast cancer-specific marker sequences and are therefore used as an indication for breast cancer. The detection and the monitoring of the quantity of breast cancer-associated autoantibodies in the patient can be used for the early detection, diagnosis and/or therapy monitoring/therapy control and for the prognosis and prediction of the risk of metastasis formation. These breast cancer-associated autoantibody profiles may be sufficiently characterised already with use of a breast cancer-specific marker sequence. In other cases, two or more breast cancer-specific marker sequences are necessary in order to indicate a breast cancer-associated autoantibody profile.

In preferred embodiments of the invention, the breast cancer-associated autoantibodies can be detected using breast cancer-specific marker sequences, which are derived from another individual, because they originate for example from a commercial cDNA bank. Preferred embodiments of the invention concern the breast cancer-associated marker sequences SEQ ID No. 1-491, SEQ ID. No. 492-982 and/or partial sequences of SEQ ID No. 1-982, and sequences that code for the proteins SEQ ID No. 983 to 1473 and/or partial sequences of these proteins.

In other preferred embodiments of the invention, the breast cancer-associated autoantibodies can be detected using breast cancer-specific marker sequences, which are derived from the same individual (autoantigen), because they originate for example from a cDNA bank produced especially for the patient or a group of patients (for example within the scope of personalised medicine). For example, homologues of the aforementioned breast cancer-specific marker sequences SEQ ID No. 1-1473 or partial sequences thereof are then used.

Autoantibodies can be formed by the patient already many years before the occurrence of the first symptoms of the disease. Early detection, diagnosis and also prognosis and (preventative) treatment would therefore be possible years before the visible outbreak of the disease. The devices and means (arrangement, array, protein array, diagnostic tool, test kit) and methods according to the invention thus enable a very early intervention compared with known methods, which considerably improves the prognosis and survival rates. Since the breast cancer-associated autoantibody profiles change during the establishment and treatment/therapy of breast cancer, the invention also enables the detection and the monitoring of breast cancer at any stage of development and treatment and also monitoring within the scope of aftercare in the case of breast cancer. The means according to the invention also allow easy handling at home by the patient themself and cost-effective routine precautionary measures for early detection and also aftercare.

In particular due to the use of antigens as specific marker sequence for breast cancer, which derive from sequences already known, for example from commercial cDNA banks, test subjects (individuals) can be tested, and, where applicable, breast cancer-associated autoantibodies present in these test subjects can be detected, even if the corresponding autoantigens are not (yet) known in these test subjects.

Different patients may have different breast cancer-associated autoantibody profiles, for example different cohorts or population groups differ from one another. Here, each patient may form one or more different breast cancer-associated autoantibodies during the course of the development of breast cancer and the progression of the disease of breast cancer, that is to say also different autoantibody profiles. In addition, the composition and/or the quantity of the formed breast cancer-associated autoantibodies may change during the course of the breast cancer development and progression of the disease, such that a quantitative evaluation is necessary. The therapy/treatment of breast cancer also leads to changes in the composition and/or the quantity of breast cancer-associated autoantibodies. The large selection of breast cancer-associated marker sequences according to the invention allows the individual compilation of breast cancer-specific marker sequences in an arrangement for individual patients, groups of patients, certain cohorts, population groups, and the like. In an individual case, the use of a breast cancer-specific marker sequence may therefore be sufficient, whereas in other cases at least two or more breast cancer-specific marker sequences have to be used together or in combination in order to produce a meaningful autoantibody profile.

Compared with other biomarkers, the detection of breast cancer-associated autoantibodies for example in the serum/plasma has the advantage of high stability and storage capability and good detectability. The presence of autoantibodies also is not subject to a circadian rhythm, and therefore the sampling is independent of the time of day, food intake and the like.

In addition, the breast cancer-associated autoantibodies can be detected with the aid of the corresponding antigens/autoantigens in known assays, such as ELISA or Western Blot, and the results can be checked for this.

In the sense of the invention, “wherein one or more breast cancer-specific marker sequence is/are selected” and “wherein one or more marker sequences for breast cancer is/are selected” means that an interaction is detected. Such an interaction is, for example, a bond, in particular a binding substance on at least one breast cancer-specific marker sequence, or, in the case that the breast cancer-specific marker sequence is a nucleic acid, for example a cDNA, the hybridisation with a suitable substance under selected conditions, in particular stringent conditions (for example as defined conventionally in J. Sambrook, E. F. Fritsch, T. Maniatis (1989), Molecular cloning: A laboratory manual, 2nd Edition, Cold Spring Habor Laboratory Press, Cold Spring Habor, USA oder Ausubel, “Current Protocols in Molecular Biology”, Green Publishing Associates and Wiley Interscience, N. Y. (1989)). One example of stringent hybridisation conditions is: hybridisation in 4×SSC at 65° C. (alternatively in 50% formamide and 4×SSC at 42° C.), followed by a number of washing steps in 0.1×SSC at 65° C. for a total of approximately one hour. An example of less stringent hybridisation conditions is hybridisation in 4×SSC at 37° C., followed by a number of washing steps in 1×SSC at room temperature.

The interaction between the bodily fluid or tissue sample from a patient and the breast cancer-specific marker sequences is preferably a protein-protein interaction.

In accordance with the invention, such substances, for example breast cancer-associated antigens, autoantigens, autoantibodies, are part of a bodily fluid, in particular blood, whole blood, blood plasma, blood serum, patient serum, urine, cerebrospinal fluid, synovial fluid or a tissue sample, for example from tumour tissue from the patient. The invention in particular relates to the use of these bodily fluids and tissue samples for early detection, diagnosis, prognosis, therapy control and aftercare.

However, in a further embodiment of the invention, the breast cancer-specific marker sequences or the substances identified from these marker sequences, for example breast cancer-associated autoantibodies, can be present in a significantly higher or lower expression rate or concentration, which is indicative of breast cancer. Here, the relative expression rates diseased/healthy of the marker sequences according to the invention for breast cancer or the substances identified from these marker sequences are determined by means of proteomics or nucleic acid blots.

The breast cancer-specific marker sequences, in a further embodiment of the invention, have a recognition signal that is addressed to the substance to be bound (for example antibody, nucleic acid). In accordance with the invention, the recognition signal for a protein is preferably an epitope and/or paratope and/or hapten, and for a cDNA is preferably a hybridisation or binding region.

The breast cancer-specific marker sequences according to the invention are detailed in Table A (RNA) and in the sequence protocol and can also be clearly identified by the respectively cited database entry (also accessible by Internet: http://www.ncbi.nlm.nih.gov/) (by means of accession no.); see also the associated sequence protocol. The clone sequences (cDNA) and protein sequences can be found in the accompanying sequence protocol.

The invention therefore also concerns the full-length sequences of the breast cancer-specific marker sequences according to the invention, more specifically as defined via the known database entry according to Table A, referred to hereinafter as SEQ 1-1473.

Furthermore, analogue embodiments to the breast cancer-specific marker sequences SEQ 1-1473, for example as presented in the claims, are therefore also included, since the SEQ 1-1473 according to the invention in turn constitute partial sequences, at least with high homology. However, the breast cancer-specific marker sequences SEQ 1-1473 are preferred in accordance with the invention.

In accordance with the invention, the marker sequences also comprise modifications of the nucleic acid sequence, in particular cDNA sequence and the corresponding amino acid sequence, such as chemical modification, such as citrullination, acetylation, phosphorylation, glycosylation or polyA strand and further modifications known as appropriate to a person skilled in the art.

The invention also relates to homologues of the breast cancer-specific marker sequences and partial sequences, for example fragments of breast cancer-specific marker sequences.

For example, homologues are nucleic acid sequences and/or protein sequences, for example homologues of SEQ ID No. 1-1473, in particular homologues of SEQ ID No. 1-491 and SEQ ID No. 492-982 that have an identity with the breast cancer-specific marker sequences of at least 70% or 80%, preferably 90% or 95%, particularly preferably 96% or 97% or more, for example 98% or 99%. In a particularly preferred embodiment of the invention, for the case in which the breast cancer-specific marker sequences are antigens, the homology in the sequence range in which the antigen-antibody or antigen-autoantibody interaction takes place, is at least 95%, preferably at least 97%, particularly preferably at least 99%. For example, mutations such as base exchange mutations, frameshift mutations, base insertion mutations, base loss mutations, point mutations and insertion mutations, are included in accordance with the invention.

The invention also relates to partial sequences of the breast cancer-specific marker sequences. Partial sequences also include fragments of the marker sequences according to the invention, and partial sequences are nucleic acids or proteins/peptides that are shortened compared with the entire nucleic acid or the entire protein/peptide. Here, the deletion may occur at the end or the ends and/or within the sequence. For example, partial sequences and/or fragments that have 50 to 100 nucleotides or 70-120 nucleotides of an entire sequence are included, for example of SEQ 1-1473. Homologues of partial sequences and fragments are also included in accordance with the invention. In a particular embodiment, the breast cancer-specific marker sequences are shortened compared with the sequences 1-1473 to such an extent that they still consist only of the binding point(s) for the breast cancer-associated autoantibody in question. In accordance with the invention, breast cancer-specific marker sequences are also included that differ from the sequences SEQ ID No. 1-1473 in that they contain one or more insertions, wherein the insertions for example are 1 to 100 or more nucleotide/amino acids long, preferably 5 to 50, particularly preferably 10 to 20 nucleotides/amino acids long and the sequences are otherwise identical however or homologous to sequences 1-1473. Partial sequences that have at least 90%, preferably at least 95%, particularly preferably 97% or 98%, of the length of the breast cancer-specific marker sequences according to the invention, SEQ IS No. 1-491, SEQ ID No. 492-982, SEQ ID No. 983-1473, are particularly preferred.

In a further embodiment, the respective breast cancer-specific marker sequence can be represented in different quantities in one or more regions in the arrangement. This allows a variation of the sensitivity. The regions may each have a totality of breast cancer-specific marker sequences, that is to say a sufficient number of different breast cancer-specific marker sequences, in particular 2, 3, 4, 5, 6, 7, 8, 9 or 10 or more different and where applicable further nucleic acids and/or proteins, in particular biomarkers.

In a particularly preferred embodiment of the invention, at least 96 to 25,000 (numerically) or more different or same breast cancer-specific marker sequences and optionally further nucleic acids and/or proteins, in particular biomarkers, are represented on the support. Further preferably, more than 2,500, particularly preferably 10,000 or more, different or same breast cancer-specific marker sequences and optionally further nucleic acids and/or proteins, in particular biomarkers, are represented on the support.

Within the scope of this invention, “arrangement” is synonymous with “array”, and, if this “array” is used to identify substances on breast cancer-specific marker sequences, this is to be understood preferably to be an “assay” or a diagnostic device. In a preferred embodiment, the arrangement is designed such that the breast cancer-specific marker sequences represented on the arrangement are present in the form of a grid on a support. Furthermore, those arrangements are preferred that permit a high-density arrangement of breast cancer-specific marker sequences, for example protein binders. The breast cancer-specific marker sequences are preferably spotted. Such high-density spotted arrangements are disclosed for example in WO 99/57311 and WO 99/57312 and can be used advantageously in a robot-supported automated high-throughput method.

Within the scope of this invention, however, the term “assay” or diagnostic device likewise comprises those embodiments of a device such as ELISA, bead-based assay, line assay, Western Blot, and immunochromatographic methods (for example what are known as lateral flow immunoassays) or similar immunological single or multiplex detection methods.

A “protein array” in the sense of this invention is the systematic arrangement of breast cancer-specific marker sequences on a solid support, wherein the breast cancer-specific marker sequences are proteins or peptides or parts thereof, and wherein the support is preferably a solid support.

The breast cancer-specific marker sequences of the arrangement are fixed on a solid support, but are preferably spotted or immobilised or even printed on, that is to say applied in a reproducible manner. One or more breast cancer-specific marker sequences can be present multiple times in the totality of all breast cancer-specific marker sequences and may be present in different quantities based on a spot. Furthermore, the breast cancer-specific marker sequences can be standardised on the solid support (for example by means of serial dilution series of, for example, human globulins as internal calibrators for data normalisation and quantitative evaluation). A standard (for example a gold standard) can also be applied to the support where necessary.

In a further embodiment, the breast cancer-specific marker sequences are present as clones. Such clones can be obtained for example by means of a cDNA expression library according to the invention (Büssow et al. 1998 (above)). In a preferred embodiment, such expression libraries containing clones are obtained using expression vectors from a cDNA expression library consisting of the cDNA marker sequences. These expression vectors preferably contain inducible promoters. The induction of the expression can be carried out for example by means of an inducer, such as IPTG. Suitable expression vectors are described in Terpe et al. (Terpe T Appl Microbiol Biotechnol. 2003 January; 60(5):523-33).

Expression libraries are known to a person skilled in the art; they can be produced in accordance with standard works, such as Sambrook et al, “Molecular Cloning, A laboratory handbook, 2nd edition (1989), CSH press, Cold Spring Harbor, N.Y. Expression libraries that are tissue-specific (for example human tissue, in particular human organs, for example from breast tissue or tissue from breast carcinoma) are furthermore preferable. Further, expression libraries that can be obtained by means of exon-trapping are also included in accordance with the invention. Instead of the term expression library, reference may also be made synonymously to an expression bank.

Protein arrays or corresponding expression libraries that do not exhibit any redundancy (what is known as a Uniclone® library) and that can be produced for example in accordance with the teaching of WO 99/57311 and WO 99/57312 are furthermore preferred. These preferred Uniclone® libraries have a high proportion of non-defective fully expressed proteins of a cDNA expression library.

Within the scope of this invention, the clones can also be, but are not limited to, transformed bacteria, recombinant phages or transformed cells of mammals, insects, fungi, yeasts or plants.

The clones are fixed, spotted or immobilised on a solid support. The invention therefore relates to an arrangement/use, wherein the breast cancer-specific marker sequences are present as clones.

In addition, the breast cancer-specific marker sequences can be present in the respective form in the form of a fusion protein, which for example contains at least one affinity epitope or “tag”. The tag may be or may contain one such as c-myc, his tag, arg tag, FLAG, alkaline phosphatase, V5 tag, T7 tag or strep tag, HAT tag, NusA, S tag, SBP tag, thioredoxin, DsbA, a fusion protein, preferably a cellulose-binding domain, green fluorescent protein, maltose-binding protein, calmodulin-binding protein, glutathione S-transferase or lacZ.

In a further preferred embodiment of the arrangement/use according to the invention, this corresponds to a grid with the dimensions of a microtiter plate (8-12 well strips, 96 wells, 384 wells or more), a silicon wafer, a chip, a mass spectrometry target or a matrix.

In a further embodiment, the invention relates to an assay or protein array for identifying and characterising a substance (for example also referred to as a hit, lead substance, candidate, active agent) for breast cancer, characterised in that an arrangement or assay according to the invention

a.) is brought into contact with at least one substance to be tested, and
b.) binding success is detected.

The substance to be tested may be any native or non-native biomolecule, a (synthetic) chemical molecule, a natural substance, a mixture or a substance library.

Once the substance to be tested has contacted a breast cancer-specific marker sequence, the binding success is evaluated, and is performed for example with use of commercially available image analysing software (GenePix Pro (Axon Laboratories), Aida (Raytest), ScanArray (Packard Bioscience).

Binding according to the invention, binding success, interactions, for example protein-protein interactions (for example protein to breast cancer-specific marker sequence, such as antigen/antibody) or corresponding “means for detecting the binding success” can be visualised for example by means of fluorescence labelling, biotinylation, radio-isotope labelling or colloid gold or latex particle labelling in the conventional manner. Bound antibodies are detected with the aid of secondary antibodies, which are labelled using commercially available reporter molecules (for example Cy, Alexa, Dyomics, FITC or similar fluorescent dyes, colloidal gold or latex particles), or with reporter enzymes, such as alkaline phosphatase, horseradish peroxidase, etc. and the corresponding colorimetric, fluorescent or chemoluminescent substrates. A readout is performed for example by means of a microarray laser scanner, a CCD camera or visually.

In a further embodiment, the invention relates to a drug/active agent or prodrug for breast cancer developed and obtainable by the use of a breast cancer-specific marker sequence according to the invention, an arrangement according to the invention, a use according to the invention, or an assay according to the invention.

The invention also relates to the use of a breast cancer-specific marker sequence selected from sequences SEQ ID No. 1- to 1473 and partial sequences of SEQ ID. No. 1-1473 with at least 90%, preferably at least 95%, of the length of SEQ ID No. 1-1473, and homologues of SEQ ID No. 1-1473 and partial sequences thereof with an identity of at least 95%, preferably at least 98% or more, to the corresponding sequences and proteins/peptides coded by the sequences SEQ ID No. 1-491, coded by the partial sequences thereof and homologues as affinity material for carrying out an apheresis or blood washing for patients with breast cancer. The invention thus relates to the use of the marker sequences according to the invention, preferably in the form of an arrangement, as affinity material for carrying out a blood washing in the broader sense, wherein substances from bodily fluids from a patient with breast cancer, such as blood or plasma, bind to the marker sequences according to the invention and consequently can be removed selectively from the bodily fluid.

The following examples explain the invention, but do not limit the invention to the examples.

The examples were carried out with use of the UNIarray technology platform on the basis of quantitative analyses of the autoantibody profiles in the serum of female patients with breast cancer. Breast cancer-associated antigens and breast cancer-associated autoantigens (biomarkers), which enable an early detection of breast cancer and/or indicate a specific form of progression (prognostic relevance), are thus to be identified systematically.

### EXAMPLE 1

Candidates for breast cancer-specific marker sequences were identified first.

In the first phase, 50 serum samples are tested for this purpose from female patients with breast carcinoma on a MACROarray (comprises approximately 10,000 different recombinant human proteins). Here, candidates for breast cancer-specific marker sequences are identified.

### EXAMPLE 2

In the subsequent test phase, these candidates for breast cancer-specific marker sequences are analysed comparatively on serum samples from 100 female patients with breast cancer and 100 female patients with benign changes of the breast or 100 healthy control female patients and characterised. As a result of this comparative analysis, marker sequences are primarily identified that interact with breast cancer-associated autoantibodies.

### EXAMPLE 3

Particularly significant biomarkers (breast cancer-specific marker sequences) are selected by means of bioinformatic analysis. The candidates for breast cancer-specific marker sequences are evaluated in terms of whether they discriminate between different test subjects (for example healthy/unhealthy)/patient groups (for example low/high risk of metastasis formation)/cohorts (for example certain past histories).

To this end, the marker sequence candidates are applied to a protein array and validated. The data evaluation is performed via statistical analyses, for example threshold value analysis, support vector machine algorithm (SVM). The sample consumption for the validation is just 50 μl/sample. In a first approach, cohorts of category I and II are selected in this way.

The protein array obtained is specific for breast cancer. This protein array comprises one or more breast cancer-specific marker sequences and identifies breast cancer-associated autoantibodies.

Cohort I: clinical finding: breast cancer-positive group (CASE group; verified via histopathological finding of the biopsy).

Cohort II: clinical finding: breast cancer-negative group (control group), age-matched.

Female patients are selected in accordance with inclusion and exclusion criteria

### Inclusion Criteria

histologically verified breast carcinoma
histologically verified non-neoplastic change
healthy age-matched control
at the moment of diagnosis (prior to operation)
prior to or during neoadjuvant and adjuvant anti-oestrogen therapy, adjuvant chemotherapy or adjuvant aromatase inhibitor therapy.

### Exclusion Criteria

age below 18 years
tumour treatment already administered

Corresponding protein arrays are developed for diagnosis, prediction of the course of therapy and prediction of metastasis formation.

### EXAMPLE 4

For the development of a protein array for the diagnosis of breast cancer, the results of the autoantibody analysis are compared with the golden standard of diagnosis and the identified marker sequences are validated (breast cancer-specific marker sequences; marker sequences for breast cancer). The results are then correlated with other clinical characteristics of breast cancer, for example tumour size and malignancy.

### EXAMPLE 5

With the development of a protein array for prediction of the course of therapy, a certain autoantibody profile or a certain signal of the protein array is correlated with the response of the breast cancer to a certain therapy. In addition, changes of the autoantibody profile are validated, even with regard to different treatment options (continuous time modelling).

### EXAMPLE 6

With the development of a protein array for the prediction of metastasis formation, breast cancer-specific marker sequences are selected that interact with breast cancer-associated autoantibodies that are suitable as indicators for metastasis formation. Due to the comparison of autoantibodies at the moment of diagnosis of female patients with and without metastasis formation, female patients who have a high metastasis risk can be identified.

Within the scope of the identification and validation of breast cancer-specific marker sequences, bioinformatic analyses can be performed. For each serum, reactivities against approximately 2,000 different antigens can be measured for this purpose by means of microarray. This data is used for a ranking of the spotted antigens with respect to their differentiation capability between healthy and diseased sera. This evaluation can be performed by means of the non-parametric Mann-Whitney test on normalised intensity data. For normalisation, an internal standard is used that is also spotted on each protein array. Since a p-value is calculated for each antigen, methods for correction of multiple testing are used. As a very conservative approach, a Bonferroni correction is performed and in addition the less restrictive False Discovery Rate (FDR) in accordance with Benjamini & Hochberg is calculated.

Furthermore, the data is used for classification of the sera. Here, different multivariate methods are used. These are methods from the statistical learning methods, such as Support Vector Machines (SVM), neuronal networks or classification trees, and a threshold value method, which is suitable both for classification and for visual representation of the data.

To avoid overfitting, a 10× cross-validation of the data is performed by way of example.

The sequences according to the invention are specified in the accompanying sequence protocol. (The clone sequences (cDNA) SEQ ID No. 1-491, the RNA sequences SEQ ID. No. 492-982 and the protein sequences SEQ ID No. 983-1473).

TABLE A
Data concerning breast cancer-specific marker
sequences (RNA) SEQ ID No. 492-982.

| SEQ | | Alias | |
|---|---|---|---|
| ID | Accession | Accession | |
| No. | No. | No. | Blast |
| 492 | gi\|261337182 | NM_182905.4 | WAS protein family homolog 1 |
| | | | [Homo sapiens] |
| 493 | gi\|16877437 | BC016965.1 | NLRP1 protein [Homo sapiens] |
| 494 | gi\|57242760 | NM_003804.3 | receptor-interacting |
| | | | serine/threonine-protein |
| | | | kinase 1 [Homo sapiens] |
| 495 | gi\|225579068 | NM_012426.4 | splicing factor 3B subunit 3 |
| | | | [Homo sapiens] |
| 496 | gi\|221136896 | NM_014786.3 | rho guanine nucleotide |
| | | | exchange factor 17 |
| | | | [Homo sapiens] |
| 497 | gi\|60218896 | NM_005748.3 | YY1-associated factor 2 |
| | | | isoform 2 [Homo sapiens] |
| 498 | gi\|157388905 | NM_017806.2 | lck-interacting transmembrane |
| | | | adapter 1 [Homo sapiens] |
| 499 | gi\|13569955 | NM_030978.1 | actin-related protein 2/3 |
| | | | complex subunit 5-like protein |
| | | | [Homo sapiens] |
| 500 | gi\|17933771 | NM_080388.1 | protein S100-A16 |
| | | | [Homo sapiens] |
| 501 | gi\|171906614 | NM_006891.3 | gamma-crystallin D |
| | | | [Homo sapiens] |
| 502 | gi\|157389013 | NM_001408.2 | cadherin EGF LAG seven-pass G- |
| | | | type receptor 2 precursor |
| | | | [Homo sapiens] |
| 503 | gi\|55956909 | NM_002430.2 | probable tumor suppressor |
| | | | protein MN1 [Homo sapiens] |
| 504 | gi\|21396481 | NM_003512.3 | histone H2A type 1-C |
| | | | [Homo sapiens] |
| 505 | gi\|77812677 | NM_006903.4 | inorganic pyrophosphatase 2, |
| | | | mitochondrial isoform 2 |
| | | | precursor [Homo sapiens] |
| 506 | gi\|41349492 | NM_013239.3 | serine/threonine-protein |
| | | | phosphatase 2A regulatory |
| | | | subunit B″ subunit beta |
| | | | [Homo sapiens] |
| 507 | gi\|56118233 | NM_181716.2 | centromere protein V |
| | | | [Homo sapiens] |
| 508 | gi\|225579128 | NM_001018024.2 | mature T-cell proliferation 1 |
| | | | neighbor protein |
| | | | [Homo sapiens] |
| 509 | gi\|334085246 | NM_014889.3 | presequence protease, |
| | | | mitochondrial isoform 2 |
| | | | precursor [Homo sapiens] |
| 510 | gi\|66932989 | NM_001018078.1 | folylpolyglutamate synthase, |
| | | | mitochondrial isoform b |
| | | | [Homo sapiens] |
| 511 | gi\|31317296 | NM_181353.1 | DNA-binding protein inhibitor |
| | | | ID-1 isoform b [Homo sapiens] |
| 512 | gi\|209413724 | NM_003692.3 | tomoregulin-1 precursor |
| | | | [Homo sapiens] |
| 513 | gi\|37181353 | AY358124.1 | SEMA5B [Homo sapiens] |
| 514 | gi\|21704284 | NM_021219.2 | junctional adhesion molecule B |
| | | | precursor [Homo sapiens] |
| 515 | gi\|254028259 | NM_182501.3 | mTERF domain-containing |
| | | | protein 2 [Homo sapiens] |
| 516 | gi\|161016768 | NM_001799.3 | cyclin-dependent kinase 7 |
| | | | [Homo sapiens] |
| 517 | gi\|180636 | M93008.1 | L-isoaspartyl/D-aspartyl |
| | | | protein carboxyl |
| | | | methyltransferase |
| | | | [Homo sapiens] |
| 518 | gi\|67782361 | NM_019030.2 | ATP-dependent RNA helicase |
| | | | DHX29 [Homo sapiens] |
| 519 | gi\|14195613 | NM_018940.2 | protocadherin beta-7 precursor |
| | | | [Homo sapiens] |
| 520 | gi\|93204870 | NM_021229.3 | netrin-4 precursor |
| | | | [Homo sapiens] |
| 521 | gi\|224967099 | NM_001734.3 | complement C1s subcomponent |
| | | | precursor [Homo sapiens] |
| 522 | gi\|16507949 | NM_014466.2 | tektin-2 [Homo sapiens] |
| 523 | gi\|75750475 | NM_001033566.1 | mitochondrial Rho GTPase 1 |
| | | | isoform 2 [Homo sapiens] |
| 524 | gi\|216548626 | NM_001142467.1 | transcription factor HES-4 |
| | | | isoform 1 [Homo sapiens] |
| 525 | gi\|38683798 | NM_033121.1 | ankyrin repeat domain- |
| | | | containing protein 13A |
| | | | [Homo sapiens] |
| 526 | gi\|285002230 | NM_001083112.2 | glycerol-3-phosphate |
| | | | dehydrogenase, mitochondrial |
| | | | precursor [Homo sapiens] |
| 527 | gi\|5454057 | NM_006278.1 | CMP-N-acetylneuraminate-beta- |
| | | | galactosamide-alpha-2,3- |
| | | | sialyltransferase 4 |
| | | | [Homo sapiens] |
| 528 | gi\|169259774 | NM_014737.2 | ras association domain- |
| | | | containing protein 2 |
| | | | [Homo sapiens] |
| 529 | gi\|126091094 | NM_001081563.1 | myotonin-protein kinase |
| | | | isoform 1 [Homo sapiens] |
| 530 | gi\|194097397 | NM_002841.3 | receptor-type tyrosine-protein |
| | | | phosphatase gamma precursor |
| | | | [Homo sapiens] |
| 531 | gi\|41350325 | NM_003791.2 | membrane-bound transcription |
| | | | factor site-1 protease |
| | | | preproprotein [Homo sapiens] |
| 532 | gi\|84508630 | NM_024419.3 | CDP-diacylglycerol--glycerol- |
| | | | 3-phosphate 3- |
| | | | phosphatidyltransferase, |
| | | | mitochondrial precursor |
| | | | [Homo sapiens] |
| 533 | gi\|187960072 | NM_006341.3 | mitotic spindle assembly |
| | | | checkpoint protein MAD2B |
| | | | [Homo sapiens] |
| 534 | gi\|76563938 | NM_015920.3 | 40S ribosomal protein S27-like |
| | | | [Homo sapiens] |
| 535 | gi\|169234806 | NM_015401.3 | histone deacetylase 7 isoform |
| | | | a [Homo sapiens] |
| 536 | gi\|120952828 | NM_001079906.1 | zinc finger protein 331 |
| | | | [Homo sapiens] |
| 537 | gi\|57222569 | NM_138477.2 | codanin-1 [Homo sapiens] |
| 538 | gi\|21361096 | NM_004125.2 | DNAJC25-GNG10 protein |
| | | | [Homo sapiens] |
| 539 | gi\|51093843 | NM_004147.3 | developmentally-regulated GTP- |
| | | | binding protein 1 |
| | | | [Homo sapiens] |
| 540 | gi\|221316745 | NM_003156.3 | stromal interaction molecule 1 |
| | | | precursor [Homo sapiens] |
| 541 | gi\|327365362 | NM_203290.2 | DNA-directed RNA polymerases I |
| | | | and III subunit RPAC1 |
| | | | [Homo sapiens] |
| 542 | gi\|114520590 | NM_032985.4 | protein transport protein |
| | | | Sec23B isoform 1 |
| | | | [Homo sapiens] |
| 543 | gi\|194018465 | NM_004454.2 | ETS translocation variant 5 |
| | | | [Homo sapiens] |
| 544 | gi\|62953137 | NM_002192.2 | inhibin beta A chain precursor |
| | | | [Homo sapiens] |
| 545 | gi\|31652260 | NM_002466.2 | myb-related protein B |
| | | | [Homo sapiens] |
| 546 | gi\|85680425 | DQ335454.1 | BS69 variant 3 [Homo sapiens] |
| 547 | gi\|218563704 | NM_025193.3 | 3 beta-hydroxysteroid |
| | | | dehydrogenase type 7 isoform a |
| | | | [Homo sapiens] |
| 548 | gi\|164419759 | NM_000107.2 | DNA damage-binding protein 2 |
| | | | [Homo sapiens] |
| 549 | gi\|114155138 | NM_153649.3 | tropomyosin alpha-3 chain |
| | | | isoform 2 [Homo sapiens] |
| 550 | gi\|215820618 | NM_014699.3 | zinc finger protein 646 |
| | | | [Homo sapiens] |
| 551 | gi\|157502196 | NM_014738.4 | hypothetical protein LOC9772 |
| | | | [Homo sapiens] |
| 552 | gi\|58331162 | NM_001009936.1 | PHD finger protein 19 isoform |
| | | | b [Homo sapiens] |
| 553 | gi\|110611232 | NM_130445.2 | collagen alpha-1(XVIII) chain |
| | | | isoform 2 precursor |
| | | | [Homo sapiens] |
| 554 | gi\|23110958 | NM_000396.2 | cathepsin K preproprotein |
| | | | [Homo sapiens] |
| 555 | gi\|150417970 | NM_003174.3 | supervillin isoform 1 |
| | | | [Homo sapiens] |
| 556 | gi\|84872172 | NM_003432.1 | zinc finger protein 131 |
| | | | [Homo sapiens] |
| 557 | gi\|54792739 | NM_014858.2 | transmembrane and coiled-coil |
| | | | domains protein 2 isoform 1 |
| | | | [Homo sapiens] |
| 558 | gi\|14589877 | NM_005864.2 | embryonal Fyn-associated |
| | | | substrate isoform 1 |
| | | | [Homo sapiens] |
| 559 | gi\|148529010 | NM_006091.3 | coronin-2B isoform 1 |
| | | | [Homo sapiens] |
| 560 | gi\|51317348 | NM_001003789.1 | rab-like protein 2B isoform 1 |
| | | | [Homo sapiens] |
| 561 | gi\|119120893 | NM_015263.2 | dmX-like protein 2 isoform 2 |
| | | | [Homo sapiens] |
| 562 | gi\|213972618 | NM_001141973.1 | probable cation-transporting |
| | | | ATPase 13A2 isoform 2 |
| | | | [Homo sapiens] |
| 563 | gi\|283135239 | NM_020155.3 | integral membrane protein |
| | | | GPR137 isoform 3 |
| | | | [Homo sapiens] |
| 564 | gi\|164565439 | NM_024571.3 | U11/U12 small nuclear |
| | | | ribonucleoprotein 25 kDa |
| | | | protein [Homo sapiens] |
| 565 | gi\|45331214 | NM_032429.1 | leucine zipper putative tumor |
| | | | suppressor 2 [Homo sapiens] |
| 566 | gi\|94721260 | NM_033133.4 | 2′,3′-cyclic-nucleotide 3′- |
| | | | phosphodiesterase |
| | | | [Homo sapiens] |
| 567 | gi\|115511050 | NM_002095.4 | transcription initiation |
| | | | factor IIE subunit beta |
| | | | [Homo sapiens] |
| 568 | gi\|156616274 | NM_002691.2 | DNA polymerase delta catalytic |
| | | | subunit [Homo sapiens] |
| 569 | gi\|38146093 | NM_005481.2 | mediator of RNA polymerase II |
| | | | transcription subunit 16 |
| | | | [Homo sapiens] |
| 570 | gi\|45597176 | NM_015043.3 | TBC1 domain family member 9B |
| | | | isoform b [Homo sapiens] |
| 571 | gi\|23238252 | NM_004377.2 | carnitine O- |
| | | | palmitoyltransferase 1, muscle |
| | | | isoform isoform a |
| | | | [Homo sapiens] |
| 572 | gi\|98986448 | NM_000496.2 | beta-crystallin B2 |
| | | | [Homo sapiens] |
| 573 | gi\|4505784 | NM_000294.1 | phosphorylase b kinase gamma |
| | | | catalytic chain, testis/liver |
| | | | isoform isoform 1 |
| | | | [Homo sapiens] |
| 574 | gi\|78482607 | NM_021129.3 | inorganic pyrophosphatase |
| | | | [Homo sapiens] |
| 575 | gi\|18027315 | AF289556.1 | unknown [Homo sapiens] |
| 576 | gi\|55770885 | NM_006537.2 | ubiquitin carboxyl-terminal |
| | | | hydrolase 3 [Homo sapiens] |
| 577 | gi\|59710102 | NM_006315.4 | polycomb group RING finger |
| | | | protein 3 [Homo sapiens] |
| 578 | gi\|2224666 | AB002361.1 | KIAA0363 [Homo sapiens] |
| 579 | gi\|33356549 | NM_182679.1 | G patch domain-containing |
| | | | protein 4 isoform 2 |
| | | | [Homo sapiens] |
| 580 | gi\|210147473 | NM_022366.2 | dimethyladenosine transferase |
| | | | 2, mitochondrial |
| | | | [Homo sapiens] |
| 581 | gi\|50346003 | NM_002626.4 | 6-phosphofructokinase, liver |
| | | | type [Homo sapiens] |
| 582 | gi\|187828433 | NM_006224.3 | phosphatidylinositol transfer |
| | | | protein alpha isoform |
| | | | [Homo sapiens] |
| 583 | gi\|53749664 | NM_005654.4 | COUP transcription factor 1 |
| | | | [Homo sapiens] |
| 584 | gi\|34485712 | NM_004663.3 | ras-related protein Rab-11A |
| | | | isoform 1 [Homo sapiens] |
| 585 | gi\|150378438 | NM_020226.3 | PR domain zinc finger protein |
| | | | 8 [Homo sapiens] |
| 586 | gi\|52694663 | NM_020799.2 | AMSH-like protease |
| | | | [Homo sapiens] |
| 587 | gi\|169646771 | NM_002064.2 | glutaredoxin-1 [Homo sapiens] |
| 588 | gi\|209969817 | NM_001540.3 | heat shock protein beta-1 |
| | | | [Homo sapiens] |
| 589 | gi\|164664491 | NM_007103.3 | NADH dehydrogenase |
| | | | [ubiquinone] flavoprotein 1, |
| | | | mitochondrial isoform 1 |
| | | | precursor [Homo sapiens] |
| 590 | gi\|48255923 | NM_021075.3 | NADH dehydrogenase |
| | | | [ubiquinone] flavoprotein 3, |
| | | | mitochondrial isoform a |
| | | | precursor [Homo sapiens] |
| 591 | gi\|8923891 | NM_018663.1 | peroxisomal membrane protein 2 |
| | | | [Homo sapiens] |
| 592 | gi\|117553583 | NM_001077523.1 | AP-3 complex subunit delta-1 |
| | | | isoform 1 [Homo sapiens] |
| 593 | gi\|119393885 | NM_014706.3 | squamous cell carcinoma |
| | | | antigen recognized by T-cells |
| | | | 3 [Homo sapiens] |
| 594 | gi\|26787964 | NM_006411.2 | 1-acyl-sn-glycerol-3-phosphate |
| | | | acyltransferase alpha |
| | | | [Homo sapiens] |
| 595 | gi\|10947033 | NM_006454.2 | max dimerization protein 4 |
| | | | [Homo sapiens] |
| 596 | gi\|187960089 | NM_015327.2 | protein SMG5 [Homo sapiens] |
| 597 | gi\|141801721 | NM_015153.2 | PHD finger protein 3 |
| | | | [Homo sapiens] |
| 598 | gi\|187761372 | NM_005744.3 | E3 ubiquitin-protein ligase |
| | | | ARIH1 [Homo sapiens] |
| 599 | gi\|102467483 | NM_017751.2 | sphingomyelin |
| | | | phosphodiesterase 4 isoform 1 |
| | | | [Homo sapiens] |
| 600 | gi\|31543086 | NM_020147.2 | THAP domain-containing protein |
| | | | 10 [Homo sapiens] |
| 601 | gi\|214010239 | NM_001142292.1 | VIP36-like protein isoform 1 |
| | | | [Homo sapiens] |
| 602 | gi\|42476160 | NM_032902.5 | protein phosphatase 1 |
| | | | regulatory subunit 16A |
| | | | [Homo sapiens] |
| 603 | gi\|333609250 | NM_001005850.2 | zinc finger protein 835 |
| | | | [Homo sapiens] |
| 604 | gi\|189011564 | NM_000038.4 | adenomatous polyposis coli |
| | | | protein isoform b |
| | | | [Homo sapiens] |
| 605 | gi\|87578391 | NM_031845.2 | microtubule-associated protein |
| | | | 2 isoform 2 [Homo sapiens] |
| 606 | gi\|19913344 | NM_006527.2 | histone RNA hairpin-binding |
| | | | protein [Homo sapiens] |
| 607 | gi\|110225356 | NM_006819.2 | stress-induced-phosphoprotein |
| | | | 1 [Homo sapiens] |
| 608 | gi\|42544225 | NM_020857.2 | vacuolar protein sorting- |
| | | | associated protein 18 homolog |
| | | | [Homo sapiens] |
| 609 | gi\|166795298 | NM_006516.2 | solute carrier family 2, |
| | | | facilitated glucose |
| | | | transporter member 1 |
| | | | [Homo sapiens] |
| 610 | gi\|211904132 | NM_014764.3 | DAZ-associated protein 2 |
| | | | isoform a [Homo sapiens] |
| 611 | gi\|148612828 | NM_001098509.1 | small G protein signaling |
| | | | modulator 2 isoform 2 |
| | | | [Homo sapiens] |
| 612 | gi\|40018634 | NM_015440.3 | monofunctional C1- |
| | | | tetrahydrofolate synthase, |
| | | | mitochondrial isoform 2 |
| | | | precursor [Homo sapiens] |
| 613 | gi\|169234785 | NM_017991.4 | hypothetical protein LOC55683 |
| | | | isoform b [Homo sapiens] |
| 614 | gi\|70778868 | NM_021933.2 | migration and invasion- |
| | | | inhibitory protein |
| | | | [Homo sapiens] |
| 615 | gi\|113204623 | NM_032193.3 | ribonuclease H2 subunit C |
| | | | [Homo sapiens] |
| 616 | gi\|65301131 | NM_138338.2 | DNA-directed RNA polymerase |
| | | | III subunit RPC8 isoform a |
| | | | [Homo sapiens] |
| 617 | gi\|221219069 | NM_145295.3 | zinc finger protein 627 |
| | | | [Homo sapiens] |
| 618 | gi\|21264342 | NM_002967.2 | scaffold attachment factor B1 |
| | | | isoform 3 [Homo sapiens] |
| 619 | gi\|197085592 | NM_006328.3 | RNA-binding protein 14 isoform |
| | | | 1 [Homo sapiens] |
| 620 | gi\|149588533 | NM_001098833.1 | ataxin-7-like protein 3 |
| | | | isoform b [Homo sapiens] |
| 621 | gi\|20270356 | NM_138802.1 | AN1-type zinc finger protein |
| | | | 2B [Homo sapiens] |
| 622 | gi\|35493700 | NM_152564.3 | vacuolar protein sorting- |
| | | | associated protein 13B isoform |
| | | | 1 [Homo sapiens] |
| 623 | gi\|142385096 | NM_001077693.2 | endothelial cell-specific |
| | | | chemotaxis regulator precursor |
| | | | [Homo sapiens] |
| 624 | gi\|71773207 | NM_000683.3 | alpha-2C adrenergic receptor |
| | | | [Homo sapiens] |
| 625 | gi\|55925575 | NM_000597.2 | insulin-like growth factor- |
| | | | binding protein 2 precursor |
| | | | [Homo sapiens] |
| 626 | gi\|71772582 | NM_001030001.1 | 40S ribosomal protein S29 |
| | | | isoform 2 [Homo sapiens] |
| 627 | gi\|18105053 | NM_014970.2 | kinesin-associated protein 3 |
| | | | isoform 1 [Homo sapiens] |
| 628 | gi\|34147578 | NM_0114338.3 | phosphatidylserine |
| | | | decarboxylase proenzyme |
| | | | [Homo sapiens] |
| 629 | gi\|12060856 | AF308303.1 | serologically defined breast |
| | | | cancer antigen NY-BR-99 |
| | | | [Homo sapiens] |
| 630 | gi\|7020624 | AK000496.1 | unnamed protein product |
| | | | [Homo sapiens] |
| 631 | gi\|209571529 | NM_182527.2 | calcium-binding protein 7 |
| | | | [Homo sapiens] |
| 632 | gi\|56090145 | NM_001005920.2 | jmjC domain-containing protein |
| | | | 8 [Homo sapiens] |
| 633 | gi\|166235894 | NM_001114089.1 | ectonucleoside triphosphate |
| | | | diphosphohydrolase 6 isoform 2 |
| | | | [Homo sapiens] |
| 634 | gi\|189571686 | NM_003731.2 | Sjoegren syndrome nuclear |
| | | | autoantigen 1 [Homo sapiens] |
| 635 | gi\|156071530 | NM_145802.3 | septin-6 isoform D |
| | | | [Homo sapiens] |
| 636 | gi\|98961132 | NM_025112.4 | zinc finger protein ZXDC |
| | | | isoform 1 [Homo sapiens] |
| 637 | gi\|66267729 | NM_052844.3 | WD repeat-containing protein |
| | | | 34 [Homo sapiens] |
| 638 | gi\|224586869 | NM_138443.3 | HAUS augmin-like complex |
| | | | subunit 1 [Homo sapiens] |
| 639 | gi\|197276595 | NM_006129.3 | bone morphogenetic protein 1 |
| | | | isoform 3 precursor |
| | | | [Homo sapiens] |
| 640 | gi\|161727457 | AB370195.1 | dihydropyrimidinase-like 2 |
| | | | long form [Homo sapiens] |
| 641 | gi\|89276761 | NM_032940.2 | DNA-directed RNA polymerase II |
| | | | subunit RPB3 [Homo sapiens] |
| 642 | gi\|197209876 | NM_003496.2 | transformation/transcription |
| | | | domain-associated protein |
| | | | [Homo sapiens] |
| 643 | gi\|119393884 | NM_005146.4 | U4/U6.U5 tri-snRNP-associated |
| | | | protein 1 [Homo sapiens] |
| 644 | gi\|38201691 | NM_007368.2 | ras GTPase-activating protein |
| | | | 3 [Homo sapiens] |
| 645 | gi\|22091458 | NM_014303.2 | pescadillo homolog |
| | | | [Homo sapiens] |
| 646 | gi\|55774983 | NM_015660.2 | GTPase IMAP family member 2 |
| | | | [Homo sapiens] |
| 647 | gi\|118498363 | NM_001079520.1 | dapper homolog 1 isoform 2 |
| | | | [Homo sapiens] |
| 648 | gi\|85062625 | NM_032377.3 | transcription elongation |
| | | | factor 1 homolog |
| | | | [Homo sapiens] |
| 649 | gi\|20357526 | NM_002074.2 | guanine nucleotide-binding |
| | | | protein G(I)/G(S)/G(T) subunit |
| | | | beta-1 [Homo sapiens] |
| 650 | gi\|194363754 | NM_000852.3 | glutathione S-transferase P |
| | | | [Homo sapiens] |
| 651 | gi\|46255046 | NM_001535.2 | protein arginine N- |
| | | | methyltransferase 2 isoform 1 |
| | | | [Homo sapiens] |
| 652 | gi\|157412269 | NM_031203.2 | heterogeneous nuclear |
| | | | ribonucleoprotein M isoform b |
| | | | [Homo sapiens] |
| 653 | gi\|223029468 | NM_006159.2 | protein kinase C-binding |
| | | | protein NELL2 isoform b |
| | | | precursor [Homo sapiens] |
| 654 | gi\|57863258 | NM_001008897.1 | T-complex protein 1 subunit |
| | | | alpha isoform b [Homo sapiens] |
| 655 | gi\|47078256 | NM_003778.3 | beta-1,4-galactosyltransferase |
| | | | 4 [Homo sapiens] |
| 656 | gi\|145611425 | NM_006814.3 | proteasome inhibitor PI31 |
| | | | subunit [Homo sapiens] |
| 657 | gi\|41406095 | NM_014003.3 | pre-mRNA-splicing factor ATP- |
| | | | dependent RNA helicase PRP16 |
| | | | [Homo sapiens] |
| 658 | gi\|178557737 | NM_006371.4 | cartilage-associated protein |
| | | | precursor [Homo sapiens] |
| 659 | gi\|91208424 | NM_012469.3 | pre-mRNA-processing factor 6 |
| | | | [Homo sapiens] |
| 660 | gi\|51871119 | NM_138401.2 | multivesicular body subunit |
| | | | 12A [Homo sapiens] |
| 661 | gi\|260898772 | NM_176812.4 | charged multivesicular body |
| | | | protein 4b [Homo sapiens] |
| 662 | gi\|142388930 | NM_152732.3 | radial spoke head protein 9 |
| | | | homolog isoform 1 |
| | | | [Homo sapiens] |
| 663 | gi\|320089575 | NM_001004333.4 | ribonuclease kappa |
| | | | [Homo sapiens] |
| 664 | gi\|76496473 | NM_000018.2 | very long-chain specific acyl- |
| | | | CoA dehydrogenase, |
| | | | mitochondrial isoform 1 |
| | | | precursor [Homo sapiens] |
| 665 | gi\|21618333 | NM_004003.2 | carnitine acetyltransferase |
| | | | isoform 2 [Homo sapiens] |
| 666 | gi\|38201713 | NM_001419.2 | ELAV-like protein 1 |
| | | | [Homo sapiens] |
| 667 | gi\|296531407 | NM_198155.3 | ES1 protein homolog, |
| | | | mitochondrial isoform Ib |
| | | | precursor [Homo sapiens] |
| 668 | gi\|45269143 | NM_012289.3 | kelch-like ECH-associated |
| | | | protein 1 [Homo sapiens] |
| 669 | gi\|41872645 | NM_014780.3 | cullin-7 isoform 2 |
| | | | [Homo sapiens] |
| 670 | gi\|42544178 | NM_012127.2 | cip1-interacting zinc finger |
| | | | protein isoform 1 |
| | | | [Homo sapiens] |
| 671 | gi\|34147607 | NM_015953.3 | nitric oxide synthase- |
| | | | interacting protein |
| | | | [Homo sapiens] |
| 672 | gi\|124256477 | NM_017974.3 | autophagy-related protein 16-1 |
| | | | isoform 2 [Homo sapiens] |
| 673 | gi\|110349768 | NM_024296.3 | coiled-coil domain-containing |
| | | | protein 28B [Homo sapiens] |
| 674 | gi\|40555764 | BC064481.1 | RNF187 protein [Homo sapiens] |
| 675 | gi\|194385887 | AK304513.1 | unnamed protein product |
| | | | [Homo sapiens] |
| 676 | gi\|194328721 | NM_001045556.2 | src-like-adapter isoform a |
| | | | [Homo sapiens] |
| 677 | gi\|31652219 | NM_133640.3 | mediator of RNA polymerase II |
| | | | transcription subunit 22 |
| | | | isoform b [Homo sapiens] |
| 678 | gi\|221136767 | NM_000371.3 | transthyretin precursor |
| | | | [Homo sapiens] |
| 679 | gi\|36287059 | NM_182709.1 | histone acetyltransferase KAT5 |
| | | | isoform 3 [Homo sapiens] |
| 680 | gi\|261399873 | NM_001009570.2 | T-complex protein 1 subunit |
| | | | eta isoform b [Homo sapiens] |
| 681 | gi\|42544141 | NM_202494.1 | PDZ domain-containing protein |
| | | | GIPC1 isoform 2 [Homo sapiens] |
| 682 | gi\|88501739 | NM_007032.5 | TRIO and F-actin-binding |
| | | | protein isoform 1 |
| | | | [Homo sapiens] |
| 683 | gi\|144953896 | NM_007184.3 | nischarin [Homo sapiens] |
| 684 | gi\|58331180 | NM_134265.2 | WD repeat and SOCS box- |
| | | | containing protein 1 isoform 2 |
| | | | [Homo sapiens] |
| 685 | gi\|217272837 | NM_006623.3 | D-3-phosphoglycerate |
| | | | dehydrogenase [Homo sapiens] |
| 686 | gi\|221136945 | NM_014502.4 | pre-mRNA-processing factor 19 |
| | | | [Homo sapiens] |
| 687 | gi\|117553614 | NM_016045.2 | protein slowmo homolog 2 |
| | | | [Homo sapiens] |
| 688 | gi\|15967154 | NM_016558.2 | SCAN domain-containing protein |
| | | | 1 isoform 1 [Homo sapiens] |
| 689 | gi\|74048433 | NM_016309.2 | leucine carboxyl |
| | | | methyltransferase 1 isoform a |
| | | | [Homo sapiens] |
| 690 | gi\|8922734 | NM_018255.1 | elongator complex protein 2 |
| | | | isoform 2 [Homo sapiens] |
| 691 | gi\|145275199 | NM_025080.3 | L-asparaginase [Homo sapiens] |
| 692 | gi\|42734378 | NM_138350.2 | THAP domain-containing protein |
| | | | 3 isoform 2 [Homo sapiens] |
| 693 | gi\|209915551 | NM_001760.3 | G1/S-specific cyclin-D3 |
| | | | isoform 2 [Homo sapiens] |
| 694 | gi\|156071497 | NM_004952.4 | ephrin-A3 precursor |
| | | | [Homo sapiens] |
| 695 | gi\|225579056 | NM_003049.3 | sodium/bile acid cotransporter |
| | | | [Homo sapiens] |
| 696 | gi\|284005308 | NM_004260.3 | ATP-dependent DNA helicase Q4 |
| | | | [Homo sapiens] |
| 697 | gi\|50726974 | NM_138501.4 | trans-2,3-enoyl-CoA reductase |
| | | | [Homo sapiens] |
| 698 | gi\|59710114 | NM_014679.3 | centrosomal protein of 57 kDa |
| | | | [Homo sapiens] |
| 699 | gi\|45643118 | NM_006117.2 | enoyl-CoA delta isomerase 2, |
| | | | mitochondrial isoform 1 |
| | | | [Homo sapiens] |
| 700 | gi\|111120323 | NM_006621.4 | putative |
| | | | adenosylhomocysteinase 2 |
| | | | isoform a [Homo sapiens] |
| 701 | gi\|33469977 | NM_182835.1 | sec1 family domain-containing |
| | | | protein 1 isoform b |
| | | | [Homo sapiens] |
| 702 | gi\|118200355 | NM_014394.2 | growth hormone-inducible |
| | | | transmembrane protein |
| | | | [Homo sapiens] |
| 703 | gi\|59850648 | NM_018443.2 | zinc finger protein 302 |
| | | | [Homo sapiens] |
| 704 | gi\|142371393 | NM_181505.2 | protein phosphatase 1 |
| | | | regulatory subunit 1B isoform |
| | | | 2 [Homo sapiens] |
| 705 | gi\|168480109 | NM_005225.2 | transcription factor E2F1 |
| | | | [Homo sapiens] |
| 706 | gi\|51102290 | NM_005395.2 | postmeiotic segregation |
| | | | increased 2-like 3 isoform 1 |
| | | | [Homo sapiens] |
| 707 | gi\|48255969 | NM_002969.3 | mitogen-activated protein |
| | | | kinase 12 [Homo sapiens] |
| 708 | gi\|170650722 | NM_014236.3 | dihydroxyacetone phosphate |
| | | | acyltransferase [Homo sapiens] |
| 709 | gi\|48527950 | NM_013365.3 | ADP-ribosylation factor- |
| | | | binding protein GGA1 isoform 1 |
| | | | [Homo sapiens] |
| 710 | gi\|213385322 | NM_001137559.1 | anaphase-promoting complex |
| | | | subunit 5 isoform b |
| | | | [Homo sapiens] |
| 711 | gi\|118498335 | NM_016520.2 | chromosome 9 open reading |
| | | | frame 78 [Homo sapiens] |
| 712 | gi\|37537686 | NM_018337.2 | zinc finger protein 444 |
| | | | [Homo sapiens] |
| 713 | gi\|68533248 | NM_018476.3 | protein BEX1 [Homo sapiens] |
| 714 | gi\|192449448 | NM_022066.3 | ubiquitin-conjugating enzyme |
| | | | E2 O [Homo sapiens] |
| 715 | gi\|12232384 | NM_022730.1 | COP9 signalosome complex |
| | | | subunit 7b [Homo sapiens] |
| 716 | gi\|201025398 | NM_024334.2 | transmembrane protein 43 |
| | | | [Homo sapiens] |
| 717 | gi\|38016923 | NM_006253.4 | 5′-AMP-activated protein |
| | | | kinase subunit beta-1 |
| | | | [Homo sapiens] |
| 718 | gi\|77404354 | NM_003908.3 | eukaryotic translation |
| | | | initiation factor 2 subunit 2 |
| | | | [Homo sapiens] |
| 719 | gi\|237649014 | NM_006824.2 | probable rRNA-processing |
| | | | protein EBP2 isoform 2 |
| | | | [Homo sapiens] |
| 720 | gi\|40068460 | NM_007284.3 | twinfilin-2 [Homo sapiens] |
| 721 | gi\|39780570 | NM_018116.2 | protein misato homolog 1 |
| | | | [Homo sapiens] |
| 722 | gi\|40068484 | NM_022834.3 | von Willebrand factor A |
| | | | domain-containing protein 1 |
| | | | isoform 1 precursor |
| | | | [Homo sapiens] |
| 723 | gi\|217416378 | NM_001142650.1 | heterogeneous nuclear |
| | | | ribonucleoprotein L-like |
| | | | isoform 2 [Homo sapiens] |
| 724 | gi\|71834871 | NM_178865.3 | serine incorporator 2 isoform |
| | | | 1 [Homo sapiens] |
| 725 | gi\|258547122 | NM_001151.3 | ADP/ATP translocase 1 |
| | | | [Homo sapiens] |
| 726 | gi\|260593722 | NM_001983.3 | DNA excision repair protein |
| | | | ERCC-1 isoform 2 |
| | | | [Homo sapiens] |
| 727 | gi\|52630340 | NM_002107.3 | histone H3.3 [Homo sapiens] |
| 728 | gi\|219879807 | NM_006325.3 | GTP-binding nuclear protein |
| | | | Ran [Homo sapiens] |
| 729 | gi\|23111017 | NM_152856.1 | RNA-binding protein 10 isoform |
| | | | 2 [Homo sapiens] |
| 730 | gi\|157785644 | NM_005876.4 | striated muscle preferentially |
| | | | expressed protein kinase |
| | | | isoform 1 [Homo sapiens] |
| 731 | gi\|52851442 | NM_006360.3 | eukaryotic translation |
| | | | initiation factor 3 subunit M |
| | | | [Homo sapiens] |
| 732 | gi\|197100772 | NM_020320.3 | probable arginyl-tRNA |
| | | | synthetase, mitochondrial |
| | | | precursor [Homo sapiens] |
| 733 | gi\|89337269 | NM_022749.5 | retinoic acid induced 16 |
| | | | [Homo sapiens] |
| 734 | gi\|47174858 | NM_025058.3 | tripartite motif-containing |
| | | | protein 46 [Homo sapiens] |
| 735 | gi\|109638742 | NM_031478.4 | hypothetical protein LOC83723 |
| | | | [Homo sapiens] |
| 736 | gi\|53729358 | NM_138361.3 | E3 ubiquitin-protein ligase |
| | | | LRSAM1 isoform 1 |
| | | | [Homo sapiens] |
| 737 | gi\|4506026 | NM_002720.1 | serine/threonine-protein |
| | | | phosphatase 4 catalytic |
| | | | subunit [Homo sapiens] |
| 738 | gi\|73858576 | NM_003254.2 | metalloproteinase inhibitor 1 |
| | | | precursor [Homo sapiens] |
| 739 | gi\|109150415 | NM_012293.1 | peroxidasin homolog precursor |
| | | | [Homo sapiens] |
| 740 | gi\|34365370 | BX641004.1 | hypothetical protein |
| | | | [Homo sapiens] |
| 741 | gi\|6005793 | NM_007213.1 | PRA1 family protein 2 |
| | | | [Homo sapiens] |
| 742 | gi\|33859677 | NM_017879.1 | zinc finger protein 416 |
| | | | [Homo sapiens] |
| 743 | gi\|149274652 | NM_020780.1 | patched domain-containing |
| | | | protein 2 [Homo sapiens] |
| 744 | gi\|50511940 | NM_133455.2 | EMI domain-containing protein |
| | | | 1 [Homo sapiens] |
| 745 | gi\|187608297 | NM_001660.3 | ADP-ribosylation factor 4 |
| | | | [Homo sapiens] |
| 746 | gi\|34335193 | NM_182720.1 | cAMP-responsive element |
| | | | modulator isoform g |
| | | | [Homo sapiens] |
| 747 | gi\|40549399 | NM_001894.4 | casein kinase I isoform |
| | | | epsilon [Homo sapiens] |
| 748 | gi\|197245333 | NM_005273.3 | guanine nucleotide-binding |
| | | | protein G(I)/G(S)/G(T) subunit |
| | | | beta-2 [Homo sapiens] |
| 749 | gi\|188497749 | NM_033500.2 | hexokinase-1 isoform HKI-td |
| | | | [Homo sapiens] |
| 750 | gi\|187828416 | NM_005586.3 | myoD family inhibitor |
| | | | [Homo sapiens] |
| 751 | gi\|38679891 | NM_006223.2 | peptidyl-prolyl cis-trans |
| | | | isomerase NIMA-interacting 4 |
| | | | isoform 1 [Homo sapiens] |
| 752 | gi\|221218992 | NM_003475.3 | ras association domain- |
| | | | containing protein 7 isoform 1 |
| | | | [Homo sapiens] |
| 753 | gi\|156071485 | NM_003680.3 | tyrosyl-tRNA synthetase, |
| | | | cytoplasmic [Homo sapiens] |
| 754 | gi\|58530880 | NM_004675.2 | GTP-binding protein Di-Ras3 |
| | | | [Homo sapiens] |
| 755 | gi\|150170698 | NM_015656.1 | kinesin-like protein KIF26A |
| | | | [Homo sapiens] |
| 756 | gi\|198386318 | NM_016188.4 | actin-like protein 6B |
| | | | [Homo sapiens] |
| 757 | gi\|49574501 | NM_016243.2 | NADH-cytochrome b5 reductase 1 |
| | | | [Homo sapiens] |
| 758 | gi\|34147350 | NM_023940.2 | ras-like protein family member |
| | | | 11B [Homo sapiens] |
| 759 | gi\|55749599 | NM_032451.1 | protein spire homolog 2 |
| | | | [Homo sapiens] |
| 760 | gi\|39992615 | BC064477.1 | PIM3 protein [Homo sapiens] |
| 761 | gi\|56118216 | NM_001008216.1 | UDP-glucose 4-epimerase |
| | | | [Homo sapiens] |
| 762 | gi\|27502382 | NM_172316.1 | homeobox protein Meis2 isoform |
| | | | h [Homo sapiens] |
| 763 | gi\|91208422 | NM_003302.2 | thyroid receptor-interacting |
| | | | protein 6 [Homo sapiens] |
| 764 | gi\|154800448 | NM_003433.3 | zinc finger protein 132 |
| | | | [Homo sapiens] |
| 765 | gi\|41872688 | NM_003660.2 | liprin-alpha-3 [Homo sapiens] |
| 766 | gi\|197382802 | NM_006769.3 | LIM domain transcription |
| | | | factor LMO4 [Homo sapiens] |
| 767 | gi\|253735774 | NM_001162383.1 | rho guanine nucleotide |
| | | | exchange factor 2 isoform 1 |
| | | | [Homo sapiens] |
| 768 | gi\|5730026 | NM_006559.1 | KH domain-containing, RNA- |
| | | | binding, signal transduction- |
| | | | associated protein 1 |
| | | | [Homo sapiens] |
| 769 | gi\|219555664 | NM_001143780.1 | solute carrier family 25 |
| | | | member 39 isoform a |
| | | | [Homo sapiens] |
| 770 | gi\|41393557 | NM_018032.3 | putative RNA-binding protein |
| | | | Luc7-like 1 isoform a [Homo |
| | | | sapiens] |
| 771 | gi\|149274623 | NM_030915.3 | protein LBH [Homo sapiens] |
| 772 | gi\|58761547 | NM_032538.1 | tau-tubulin kinase 1 |
| | | | [Homo sapiens] |
| 773 | gi\|34147461 | NM_033414.2 | zinc finger protein 622 |
| | | | [Homo sapiens] |
| 774 | gi\|304555613 | NM_152743.3 | BRCA1-associated ATM activator |
| | | | 1 [Homo sapiens] |
| 775 | gi\|229608893 | NM_181723.2 | EF-hand domain-containing |
| | | | family member A2 |
| | | | [Homo sapiens] |
| 776 | gi\|119964727 | NM_152783.3 | D-2-hydroxyglutarate |
| | | | dehydrogenase, mitochondrial |
| | | | precursor [Homo sapiens] |
| 777 | gi\|109148541 | NM_001605.2 | alanyl-tRNA synthetase, |
| | | | cytoplasmic [Homo sapiens] |
| 778 | gi\|83700234 | NM_001967.3 | eukaryotic initiation factor |
| | | | 4A-II [Homo sapiens] |
| 779 | gi\|105990523 | NM_000182.4 | trifunctional enzyme subunit |
| | | | alpha, mitochondrial precursor |
| | | | [Homo sapiens] |
| 780 | gi\|18201902 | NM_002109.3 | histidyl-tRNA synthetase, |
| | | | cytoplasmic [Homo sapiens] |
| 781 | gi\|78190460 | NM_000984.5 | 60S ribosomal protein L23a |
| | | | [Homo sapiens] |
| 782 | gi\|78190459 | NM_000978.3 | 60S ribosomal protein L23 |
| | | | [Homo sapiens] |
| 783 | gi\|45446742 | NM_007372.2 | ATP-dependent RNA helicase |
| | | | DDX42 [Homo sapiens] |
| 784 | gi\|61102726 | NM_015315.3 | la-related protein 1 isoform 1 |
| | | | [Homo sapiens] |
| 785 | gi\|112382376 | NM_014501.2 | ubiquitin-conjugating enzyme |
| | | | E2 S [Homo sapiens] |
| 786 | gi\|82830423 | NM_001037533.1 | GON-4-like protein isoform a |
| | | | [Homo sapiens] |
| 787 | gi\|110227844 | NM_024112.3 | hypothetical protein LOC79095 |
| | | | [Homo sapiens] |
| 788 | gi\|149363677 | NM_199287.2 | coiled-coil domain-containing |
| | | | protein 137 [Homo sapiens] |
| 789 | gi\|194353969 | NM_000681.3 | alpha-2A adrenergic receptor |
| | | | [Homo sapiens] |
| 790 | gi\|11038670 | NM_004339.2 | pituitary tumor-transforming |
| | | | gene 1 protein-interacting |
| | | | protein precursor |
| | | | [Homo sapiens] |
| 791 | gi\|87159810 | NM_004357.4 | CD151 antigen [Homo sapiens] |
| 792 | gi\|166235161 | NM_001839.3 | calponin-3 [Homo sapiens] |
| 793 | gi\|46411186 | NM_005234.3 | nuclear receptor subfamily 2 |
| | | | group F member 6 |
| | | | [Homo sapiens] |
| 794 | gi\|156071503 | NM_006908.4 | ras-related C3 botulinum toxin |
| | | | substrate 1 isoform Rac1 |
| | | | [Homo sapiens] |
| 795 | gi\|37577134 | NM_003348.3 | ubiquitin-conjugating enzyme |
| | | | E2 N [Homo sapiens] |
| 796 | gi\|37594440 | NM_003437.2 | zinc finger protein 136 |
| | | | [Homo sapiens] |
| 797 | gi\|197333754 | NM_006764.4 | interferon-related |
| | | | developmental regulator 2 |
| | | | [Homo sapiens] |
| 798 | gi\|31652256 | NM_005461.3 | transcription factor MafB |
| | | | [Homo sapiens] |
| 799 | gi\|114796625 | NM_013356.2 | monocarboxylate transporter 3 |
| | | | [Homo sapiens] |
| 800 | gi\|18496982 | NM_015526.1 | CAP-Gly domain-containing |
| | | | linker protein 3 |
| | | | [Homo sapiens] |
| 801 | gi\|323635445 | NM_020394.4 | zinc finger protein 695 |
| | | | isoform 1 [Homo sapiens] |
| 802 | gi\|197245455 | NM_022574.4 | PERQ amino acid-rich with GYF |
| | | | domain-containing protein 1 |
| | | | [Homo sapiens] |
| 803 | gi\|186928846 | NM_032830.2 | cirhin [Homo sapiens] |
| 804 | gi\|62241010 | NM_005163.2 | RAC-alpha serine/threonine- |
| | | | protein kinase [Homo sapiens] |
| 805 | gi\|38372939 | NM_001185.2 | zinc-alpha-2-glycoprotein |
| | | | precursor [Homo sapiens] |
| 806 | gi\|21735620 | NM_005918.2 | malate dehydrogenase, |
| | | | mitochondrial precursor |
| | | | [Homo sapiens] |
| 807 | gi\|115387093 | NM_003000.2 | succinate dehydrogenase |
| | | | [ubiquinone] iron-sulfur |
| | | | subunit, mitochondrial |
| | | | precursor [Homo sapiens] |
| 808 | gi\|54607088 | NM_001005914.1 | semaphorin-3B isoform 2 |
| | | | precursor [Homo sapiens] |
| 809 | gi\|56550050 | NM_006590.2 | U4/U6.U5 tri-snRNP-associated |
| | | | protein 2 [Homo sapiens] |
| 810 | gi\|38027945 | NM_006833.4 | COP9 signalosome complex |
| | | | subunit 6 [Homo sapiens] |
| 811 | gi\|57617038 | NM_015140.2 | tubulin--tyrosine ligase-like |
| | | | protein 12 [Homo sapiens] |
| 812 | gi\|110227859 | NM_016305.2 | SS18-like protein 2 |
| | | | [Homo sapiens] |
| 813 | gi\|222831567 | NM_033082.3 | SAP domain-containing |
| | | | ribonucleoprotein |
| | | | [Homo sapiens] |
| 814 | gi\|40548381 | NM_199249.1 | multidrug resistance-related |
| | | | protein [Homo sapiens] |
| 815 | gi\|89886471 | NM_206538.2 | hematopoietic signal peptide- |
| | | | containing isoform 2 |
| | | | [Homo sapiens] |
| 816 | gi\|19913440 | NM_002149.2 | hippocalcin-like protein 1 |
| | | | [Homo sapiens] |
| 817 | gi\|316983123 | NM_021029.5 | 60S ribosomal protein L36a |
| | | | isoform a [Homo sapiens] |
| 818 | gi\|55925657 | NM_002997.4 | syndecan-1 precursor |
| | | | [Homo sapiens] |
| 819 | gi\|46877103 | NM_003367.2 | upstream stimulatory factor 2 |
| | | | isoform 1 [Homo sapiens] |
| 820 | gi\|88853068 | NM_000638.3 | vitronectin precursor |
| | | | [Homo sapiens] |
| 821 | gi\|4503824 | NM_003505.1 | frizzled-1 precursor |
| | | | [Homo sapiens] |
| 822 | gi\|133908634 | NM_012109.2 | transmembrane protein 59-like |
| | | | precursor [Homo sapiens] |
| 823 | gi\|151101234 | NM_012461.2 | TERF1-interacting nuclear |
| | | | factor 2 isoform 2 |
| | | | [Homo sapiens] |
| 824 | gi\|221316691 | NM_020309.3 | vesicular glutamate |
| | | | transporter 1 [Homo sapiens] |
| 825 | gi\|165932369 | NM_024582.4 | protocadherin Fat 4 precursor |
| | | | [Homo sapiens] |
| 826 | gi\|52353305 | NM_001005210.1 | leucine-rich repeat-containing |
| | | | protein 55 [Homo sapiens] |
| 827 | gi\|194378373 | AK294825.1 | unnamed protein product |
| | | | [Homo sapiens] |
| 828 | gi\|32454740 | NM_001235.2 | serpin H1 precursor |
| | | | [Homo sapiens] |
| 829 | gi\|33598925 | NM_005506.2 | lysosome membrane protein 2 |
| | | | isoform 1 [Homo sapiens] |
| 830 | gi\|18201904 | NM_000175.2 | glucose-6-phosphate isomerase |
| | | | isoform 2 [Homo sapiens] |
| 831 | gi\|161169039 | NM_004548.2 | NADH dehydrogenase |
| | | | [ubiquinone] 1 beta subcomplex |
| | | | subunit 10 [Homo sapiens] |
| 832 | gi\|141801911 | NM_003295.2 | translationally-controlled |
| | | | tumor protein [Homo sapiens] |
| 833 | gi\|23397695 | NM_152925.1 | copine-1 isoform a |
| | | | [Homo sapiens] |
| 834 | gi\|197313723 | NM_005736.3 | alpha-centractin |
| | | | [Homo sapiens] |
| 835 | gi\|34147665 | NM_006374.3 | serine/threonine-protein |
| | | | kinase 25 [Homo sapiens] |
| 836 | gi\|54112115 | NM_006802.2 | splicing factor 3A subunit 3 |
| | | | [Homo sapiens] |
| 837 | gi\|63082031 | NM_015089.2 | cullin-9 [Homo sapiens] |
| 838 | gi\|41872442 | NM_199368.1 | short transient receptor |
| | | | potential channel 4-associated |
| | | | protein isoform b |
| | | | [Homo sapiens] |
| 839 | gi\|197100212 | NM_001610.2 | lysosomal acid phosphatase |
| | | | isoform 1 precursor |
| | | | [Homo sapiens] |
| 840 | gi\|169636438 | NM_000078.2 | cholesteryl ester transfer |
| | | | protein precursor |
| | | | [Homo sapiens] |
| 841 | gi\|169259765 | NM_001514.5 | transcription initiation |
| | | | factor IIB [Homo sapiens] |
| 842 | gi\|63253297 | NM_003132.2 | spermidine synthase |
| | | | [Homo sapiens] |
| 843 | gi\|37577149 | NM_016453.2 | NCR-interacting protein with |
| | | | SH3 domain isoform 1 |
| | | | [Homo sapiens] |
| 844 | gi\|37622893 | NM_194460.1 | RING finger protein 126 |
| | | | [Homo sapiens] |
| 845 | gi\|29171685 | NM_030768.2 | integrin-linked kinase- |
| | | | associated serine/threonine |
| | | | phosphatase 2C [Homo sapiens] |
| 846 | gi\|14150140 | NM_032346.1 | programmed cell death protein |
| | | | 2-like [Homo sapiens] |
| 847 | gi\|119120876 | NM_133474.2 | zinc finger protein 721 |
| | | | [Homo sapiens] |
| 848 | gi\|257471022 | NM_004930.3 | F-actin-capping protein |
| | | | subunit beta isoform 1 |
| | | | [Homo sapiens] |
| 849 | gi\|19718776 | NM_004111.4 | flap endonuclease 1 |
| | | | [Homo sapiens] |
| 850 | gi\|24797084 | NM_002265.4 | importin subunit beta-1 |
| | | | [Homo sapiens] |
| 851 | gi\|221316755 | NM_000425.3 | neural cell adhesion molecule |
| | | | L1 isoform 1 precursor |
| | | | [Homo sapiens] |
| 852 | gi\|171543862 | NM_000289.4 | 6-phosphofructokinase, muscle |
| | | | type isoform 2 [Homo sapiens] |
| 853 | gi\|4505940 | NM_000938.1 | DNA-directed RNA polymerase II |
| | | | subunit RPB2 [Homo sapiens] |
| 854 | gi\|156631004 | NM_002812.4 | 26S proteasome non-ATPase |
| | | | regulatory subunit 8 |
| | | | [Homo sapiens] |
| 855 | gi\|51477705 | NM_003078.3 | SWI/SNF-related matrix- |
| | | | associated actin-dependent |
| | | | regulator of chromatin |
| | | | subfamily D member 3 isoform 1 |
| | | | [Homo sapiens] |
| 856 | gi\|38505154 | NM_003195.4 | transcription elongation |
| | | | factor A protein 2 isoform a |
| | | | [Homo sapiens] |
| 857 | gi\|63162571 | NM_005998.3 | T-complex protein 1 subunit |
| | | | gamma isoform a [Homo sapiens] |
| 858 | gi\|55769586 | NM_003703.1 | nucleolar protein 14 |
| | | | [Homo sapiens] |
| 859 | gi\|95147537 | NM_006051.3 | amyloid beta A4 precursor |
| | | | protein-binding family B |
| | | | member 3 isoform d |
| | | | [Homo sapiens] |
| 860 | gi\|8922332 | NM_018049.1 | pleckstrin homology domain- |
| | | | containing family J member 1 |
| | | | [Homo sapiens] |
| 861 | gi\|32484989 | NM_018639.3 | WD repeat and SOCS box- |
| | | | containing protein 2 |
| | | | [Homo sapiens] |
| 862 | gi\|215599267 | NM_032039.2 | protein ITFG3 [Homo sapiens] |
| 863 | gi\|21362049 | NM_032357.2 | coiled-coil domain-containing |
| | | | protein 115 [Homo sapiens] |
| 864 | gi\|160420327 | NM_194279.2 | iron-sulfur cluster assembly 2 |
| | | | homolog, mitochondrial |
| | | | precursor [Homo sapiens] |
| 865 | gi\|111494227 | NM_001418.3 | eukaryotic translation |
| | | | initiation factor 4 gamma 2 |
| | | | isoform 1 [Homo sapiens] |
| 866 | gi\|61835203 | NM_001013436.1 | 3-mercaptopyruvate |
| | | | sulfurtransferase isoform 2 |
| | | | [Homo sapiens] |
| 867 | gi\|39725687 | NM_005002.3 | NADH dehydrogenase |
| | | | [ubiquinone] 1 alpha |
| | | | subcomplex subunit 9, |
| | | | mitochondrial precursor |
| | | | [Homo sapiens] |
| 868 | gi\|106049291 | NM_022172.2 | pyruvate carboxylase, |
| | | | mitochondrial precursor |
| | | | [Homo sapiens] |
| 869 | gi\|156416002 | NM_004168.2 | succinate dehydrogenase |
| | | | [ubiquinone] flavoprotein |
| | | | subunit, mitochondrial |
| | | | precursor [Homo sapiens] |
| 870 | gi\|209413761 | NM_017586.2 | calcium channel flower homolog |
| | | | isoform a [Homo sapiens] |
| 871 | gi\|166197689 | NM_001114090.1 | ephexin-1 isoform 2 |
| | | | [Homo sapiens] |
| 872 | gi\|75677342 | NM_015544.2 | transmembrane protein 98 |
| | | | [Homo sapiens] |
| 873 | gi\|187829451 | NM_001127231.1 | autism susceptibility gene 2 |
| | | | protein isoform 2 |
| | | | [Homo sapiens] |
| 874 | gi\|218751872 | NM_018645.4 | transcription cofactor HES-6 |
| | | | isoform a [Homo sapiens] |
| 875 | gi\|91807118 | NM_025141.3 | TM2 domain-containing protein |
| | | | 3 isoform b [Homo sapiens] |
| 876 | gi\|114520614 | NM_001954.4 | epithelial discoidin domain- |
| | | | containing receptor 1 isoform |
| | | | 1 precursor [Homo sapiens] |
| 877 | gi\|186659502 | NM_031263.2 | heterogeneous nuclear |
| | | | ribonucleoprotein K isoform a |
| | | | [Homo sapiens] |
| 878 | gi\|313760623 | NM_000442.4 | platelet endothelial cell |
| | | | adhesion molecule precursor |
| | | | [Homo sapiens] |
| 879 | gi\|34335279 | NM_002811.3 | 26S proteasome non-ATPase |
| | | | regulatory subunit 7 |
| | | | [Homo sapiens] |
| 880 | gi\|30581139 | NM_006263.2 | proteasome activator complex |
| | | | subunit 1 isoform 1 |
| | | | [Homo sapiens] |
| 881 | gi\|78191800 | NM_000997.4 | 60S ribosomal protein L37 |
| | | | [Homo sapiens] |
| 882 | gi\|197927095 | NM_001030.4 | 40S ribosomal protein S27 |
| | | | [Homo sapiens] |
| 883 | gi\|21396499 | NM_003609.2 | HIRA-interacting protein 3 |
| | | | [Homo sapiens] |
| 884 | gi\|25777595 | NM_003648.2 | diacylglycerol kinase delta |
| | | | isoform 1 [Homo sapiens] |
| 885 | gi\|52486264 | NM_006029.4 | paraneoplastic antigen Ma1 |
| | | | [Homo sapiens] |
| 886 | gi\|57242773 | NM_014680.2 | hypothetical protein LOC9703 |
| | | | precursor [Homo sapiens] |
| 887 | gi\|151108508 | NM_014859.4 | rho GTPase-activating protein |
| | | | 44 [Homo sapiens] |
| 888 | gi\|104876422 | NM_006040.2 | heparan sulfate glucosamine 3- |
| | | | O-sulfotransferase 4 |
| | | | [Homo sapiens] |
| 889 | gi\|110349723 | NM_021267.3 | LAG1 longevity assurance |
| | | | homolog 1 isoform 1 |
| | | | [Homo sapiens] |
| 890 | gi\|166795249 | NM_006845.3 | kinesin-like protein KIF2C |
| | | | [Homo sapiens] |
| 891 | gi\|38570153 | NM_012279.2 | zinc finger protein 346 |
| | | | [Homo sapiens] |
| 892 | gi\|156602659 | NM_018206.4 | vacuolar protein sorting- |
| | | | associated protein 35 |
| | | | [Homo sapiens] |
| 893 | gi\|17978480 | NM_080413.1 | vacuolar protein sorting- |
| | | | associated protein 16 homolog |
| | | | isoform 3 [Homo sapiens] |
| 894 | gi\|40807483 | NM_030815.2 | p53 and DNA damage-regulated |
| | | | protein 1 [Homo sapiens] |
| 895 | gi\|47132623 | NM_145798.2 | oxysterol-binding protein- |
| | | | related protein 7 |
| | | | [Homo sapiens] |
| 896 | gi\|86198309 | NM_001039355.1 | mitochondrial |
| | | | carnitine/acylcarnitine |
| | | | carrier protein CACL |
| | | | [Homo sapiens] |
| 897 | gi\|194097322 | NM_004092.3 | enoyl-CoA hydratase, |
| | | | mitochondrial precursor |
| | | | [Homo sapiens] |
| 898 | gi\|10434001 | AK022548.1 | unnamed protein product |
| | | | [Homo sapiens] |
| 899 | gi\|54792063 | NM_003352.4 | small ubiquitin-related |
| | | | modifier 1 isoform a precursor |
| | | | [Homo sapiens] |
| 900 | gi\|83281439 | NM_003757.2 | eukaryotic translation |
| | | | initiation factor 3 subunit I |
| | | | [Homo sapiens] |
| 901 | gi\|33469915 | NM_003906.3 | 80 kDa MCM3-associated protein |
| | | | [Homo sapiens] |
| 902 | gi\|195972858 | NM_004228.5 | cytohesin-2 isoform 2 |
| | | | [Homo sapiens] |
| 903 | gi\|42716281 | NM_013242.2 | transcription factor IIB |
| | | | [Homo sapiens] |
| 904 | gi\|56676378 | NM_016004.2 | intraflagellar transport |
| | | | protein 52 homolog |
| | | | [Homo sapiens] |
| 905 | gi\|261490711 | NM_138399.4 | transmembrane protein 44 |
| | | | isoform a [Homo sapiens] |
| 906 | gi\|41327758 | NM_001697.2 | ATP synthase subunit O, |
| | | | mitochondrial precursor |
| | | | [Homo sapiens] |
| 907 | gi\|124053441 | NM_000934.3 | alpha-2-antiplasmin isoform a |
| | | | precursor [Homo sapiens] |
| 908 | gi\|47132573 | NM_002733.3 | 5′-AMP-activated protein |
| | | | kinase subunit gamma-1isoform |
| | | | 1 [Homo sapiens] |
| 909 | gi\|47132588 | NM_002741.3 | serine/threonine-protein |
| | | | kinase N1 isoform 2 |
| | | | [Homo sapiens] |
| 910 | gi\|257196241 | NM_001063.3 | serotransferrin precursor |
| | | | [Homo sapiens] |
| 911 | gi\|205277461 | NM_001064.2 | transketolase [Homo sapiens] |
| 912 | gi\|253795505 | NM_004699.2 | XAP-5 protein [Homo sapiens] |
| 913 | gi\|115527096 | NM_006035.3 | serine/threonine-protein |
| | | | kinase MRCK beta |
| | | | [Homo sapiens] |
| 914 | gi\|68161505 | NM_005718.3 | actin-related protein 2/3 |
| | | | complex subunit 4 isoform a |
| | | | [Homo sapiens] |
| 915 | gi\|194097343 | NM_001114107.2 | PDZ and LIM domain protein 3 |
| | | | isoform b [Homo sapiens] |
| 916 | gi\|32307179 | NM_016139.2 | coiled-coil-helix-coiled-coil- |
| | | | helix domain-containing |
| | | | protein 2, mitochondrial |
| | | | precursor [Homo sapiens] |
| 917 | gi\|170932470 | NM_024650.3 | putative uncharacterized |
| | | | protein C11orf80 |
| | | | [Homo sapiens] |
| 918 | gi\|221218971 | NM_031209.2 | queuine tRNA- |
| | | | ribosyltransferase |
| | | | [Homo sapiens] |
| 919 | gi\|54607109 | NM_174929.2 | zinc finger MIZ domain- |
| | | | containing protein 2 isoform 2 |
| | | | [Homo sapiens] |
| 920 | gi\|58761495 | NM_001011724.1 | heterogeneous nuclear |
| | | | ribonucleoprotein A1-like 2 |
| | | | [Homo sapiens] |
| 921 | gi\|117956372 | NM_001077685.1 | arf-GAP with GTPase, ANK |
| | | | repeat and PH domain- |
| | | | containing protein 7 |
| | | | [Homo sapiens] |
| 922 | gi\|50301237 | NM_000637.2 | glutathione reductase, |
| | | | mitochondrial isoform 1 |
| | | | precursor [Homo sapiens] |
| 923 | gi\|48255890 | NM_001001329.1 | glucosidase 2 subunit beta |
| | | | isoform 2 [Homo sapiens] |
| 924 | gi\|42544245 | NM_003009.2 | selenoprotein W [Homo sapiens] |
| 925 | gi\|6102857 | AL122064.1 | hypothetical protein [Homo |
| | | | sapiens] |
| 926 | gi\|186928845 | NM_032476.3 | 28S ribosomal protein S6, |
| | | | mitochondrial [Homo sapiens] |
| 927 | gi\|19913436 | NM_001694.2 | V-type proton ATPase 16 kDa |
| | | | proteolipid subunit |
| | | | [Homo sapiens] |
| 928 | gi\|205277389 | NM_004082.3 | dynactin subunit 1 isoform 1 |
| | | | [Homo sapiens] |
| 929 | gi\|55750040 | NM_001940.3 | atrophin-1 [Homo sapiens] |
| 930 | gi\|156071491 | NM_002086.4 | growth factor receptor-bound |
| | | | protein 2 isoform 1 |
| | | | [Homo sapiens] |
| 931 | gi\|194018519 | NM_002094.3 | eukaryotic peptide chain |
| | | | release factor GTP-binding |
| | | | subunit ERF3A isoform 1 |
| | | | [Homo sapiens] |
| 932 | gi\|41399283 | NM_002156.4 | 60 kDa heat shock protein, |
| | | | mitochondrial [Homo sapiens] |
| 933 | gi\|116829963 | NM_002256.3 | metastasis-suppressor KiSS-1 |
| | | | [Homo sapiens] |
| 934 | gi\|153945727 | NM_005909.3 | microtubule-associated protein |
| | | | 1B [Homo sapiens] |
| 935 | gi\|189409157 | NM_016841.3 | microtubule-associated protein |
| | | | tau isoform 4 [Homo sapiens] |
| 936 | gi\|14043021 | NM_004990.2 | methionyl-tRNA synthetase, |
| | | | cytoplasmic [Homo sapiens] |
| 937 | gi\|32307122 | NM_004576.2 | serine/threonine-protein |
| | | | phosphatase 2A 55 kDa |
| | | | regulatory subunit B beta |
| | | | isoform isoform a |
| | | | [Homo sapiens] |
| 938 | gi\|22538466 | NM_002796.2 | proteasome subunit beta type-4 |
| | | | [Homo sapiens] |
| 939 | gi\|48762925 | NM_005049.2 | periodic tryptophan protein 2 |
| | | | homolog [Homo sapiens] |
| 940 | gi\|71164881 | NM_001013.3 | 40S ribosomal protein S9 |
| | | | [Homo sapiens] |
| 941 | gi\|171846267 | NM_005726.4 | elongation factor Ts, |
| | | | mitochondrial isoform 2 |
| | | | precursor [Homo sapiens] |
| 942 | gi\|23238209 | NM_005731.2 | actin-related protein 2/3 |
| | | | complex subunit 2 |
| | | | [Homo sapiens] |
| 943 | gi\|14971416 | NM_005762.2 | transcription intermediary |
| | | | factor 1-beta [Homo sapiens] |
| 944 | gi\|207028746 | NM_006096.3 | protein NDRG1 [Homo sapiens] |
| 945 | gi\|22907051 | NM_006409.2 | actin-related protein 2/3 |
| | | | complex subunit 1A isoform 1 |
| | | | [Homo sapiens] |
| 946 | gi\|11055016 | AF142569.1 | hypothetical protein SBBI23 |
| | | | [Homo sapiens] |
| 947 | gi\|112421107 | NM_015125.3 | protein capicua homolog |
| | | | [Homo sapiens] |
| 948 | gi\|56676386 | NM_007364.2 | transmembrane emp24 domain- |
| | | | containing protein 3 precursor |
| | | | [Homo sapiens] |
| 949 | gi\|56676313 | NM_002568.3 | polyadenylate-binding protein |
| | | | 1 [Homo sapiens] |
| 950 | gi\|24431995 | NM_020145.2 | endophilin-B2 [Homo sapiens] |
| 951 | gi\|141802780 | NM_052868.2 | immunoglobulin superfamily |
| | | | member 8 [Homo sapiens] |
| 952 | gi\|17149812 | NM_057161.2 | kelch domain-containing |
| | | | protein 3 isoform 1 |
| | | | [Homo sapiens] |
| 953 | gi\|28274700 | NM_145806.2 | zinc finger protein 511 |
| | | | [Homo sapiens] |
| 954 | gi\|4505488 | NM_002539.1 | ornithine decarboxylase |
| | | | [Homo sapiens] |
| 955 | gi\|78217389 | NM_001004.3 | 60S acidic ribosomal protein |
| | | | P2 [Homo sapiens] |
| 956 | gi\|62739176 | NM_002950.3 | dolichyl- |
| | | | diphosphooligosaccharide-- |
| | | | protein glycosyltransferase |
| | | | subunit 1 precursor |
| | | | [Homo sapiens] |
| 957 | gi\|194595508 | NM_001130438.1 | spectrin alpha chain, brain |
| | | | isoform 1 [Homo sapiens] |
| 958 | gi\|13236578 | NM_024330.1 | long-chain fatty acid |
| | | | transport protein 3 |
| | | | [Homo sapiens] |
| 959 | gi\|215277016 | NM_001142370.1 | tyrosine-protein phosphatase |
| | | | non-receptor type 18 isoform 2 |
| | | | [Homo sapiens] |
| 960 | gi\|194018569 | NM_017918.4 | coiled-coil domain-containing |
| | | | protein 109B [Homo sapiens] |
| 961 | gi\|256818771 | NM_018285.3 | U3 small nucleolar |
| | | | ribonucleoprotein protein IMP3 |
| | | | [Homo sapiens] |
| 962 | gi\|209976985 | NM_018386.2 | PCI domain-containing protein |
| | | | 2 [Homo sapiens] |
| 963 | gi\|113722119 | NM_032119.3 | G-protein coupled receptor 98 |
| | | | precursor [Homo sapiens] |
| 964 | gi\|10438604 | AK025936.1 | unnamed protein product |
| | | | [Homo sapiens] |
| 965 | gi\|50234894 | NM_173473.2 | anaphase-promoting complex |
| | | | subunit 16 isoform 1 |
| | | | [Homo sapiens] |
| 966 | gi\|207028465 | NM_005566.3 | L-lactate dehydrogenase A |
| | | | chain isoform 1 [Homo sapiens] |
| 967 | gi\|194440683 | NM_002337.2 | alpha-2-macroglobulin |
| | | | receptor-associated protein |
| | | | precursor [Homo sapiens] |
| 968 | gi\|33356548 | NM_002388.3 | DNA replication licensing |
| | | | factor MCM3 [Homo sapiens] |
| 969 | gi\|28193181 | BX248001.1 | unnamed protein product |
| | | | [Homo sapiens] |
| 970 | gi\|33598947 | NM_002660.2 | 1-phosphatidylinositol-4,5- |
| | | | bisphosphate phosphodiesterase |
| | | | gamma-1 isoform a |
| | | | [Homo sapiens] |
| 971 | gi\|223468675 | NM_021975.3 | transcription factor p65 |
| | | | isoform 1 [Homo sapiens] |
| 972 | gi\|49640008 | NM_003316.3 | E3 ubiquitin-protein ligase |
| | | | TTC3 [Homo sapiens] |
| 973 | gi\|166064032 | NM_145735.2 | rho guanine nucleotide |
| | | | exchange factor 7 isoform b |
| | | | [Homo sapiens] |
| 974 | gi\|83716023 | NM_017596.2 | kinesin-like protein KIF21B |
| | | | [Homo sapiens] |
| 975 | gi\|109148507 | NM_017670.2 | ubiquitin thioesterase OTUB1 |
| | | | [Homo sapiens] |
| 976 | gi\|11034854 | NM_020644.1 | transmembrane protein 9B |
| | | | precursor [Homo sapiens] |
| 977 | gi\|50345990 | NM_001001975.1 | ATP synthase subunit delta, |
| | | | mitochondrial precursor |
| | | | [Homo sapiens] |
| 978 | gi\|145580576 | NM_001329.2 | C-terminal-binding protein 2 |
| | | | isoform 1 [Homo sapiens] |
| 979 | gi\|226958667 | NM_004107.4 | IgG receptor FcRn large |
| | | | subunit p51 precursor |
| | | | [Homo sapiens] |
| 980 | gi\|51476153 | CR749210.1 | hypothetical protein |
| | | | [Homo sapiens] |
| 981 | gi\|193082959 | NM_014487.4 | zinc finger protein 330 |
| | | | [Homo sapiens] |
| 982 | gi\|156416004 | NM_014186.3 | COMM domain-containing protein |
| | | | 9 isoform 1 [Homo sapiens] |

1-13. (canceled)

14. A method for identifying marker sequences for breast cancer, comprising
a. identifying marker sequence candidates for breast cancer in that a support, on which at least 1,000 different proteins are immobilised, is brought into contact with a serum sample from a patient with breast cancer and proteins that demonstrate an interaction with the serum are identified (marker sequence candidates), and
b. determining the interaction of one or more marker sequence candidates from a. with the serum from female patients with breast cancer is compared with
the interaction of the marker sequence candidate(s) from a. with the serum from female patients with benign changes and
the interaction of the marker sequence candidate(s) from a. with the serum of healthy control individuals, and
c. identifying marker sequences that demonstrate an interaction with the serum from female patients with breast cancer that is different compared with the interaction with the serum from female patients with benign changes and the serum from healthy control individuals, and wherein the evaluation is performed by means of statistical analysis.

15. The method according to claim 14, wherein the marker sequences are specific for breast cancer with a high risk of metastasis formation.

16. A marker sequence for breast cancer obtainable by a method according to claim 14 and selected from the sequences comprising SEQ ID No. 1-1473 and partial sequences of SEQ ID No. 1-1473 with at least 90%, preferably 95%, of the length of the sequences SEQ ID No. 1-1473 and homologues of SEQ ID No. 1-1473 and partial sequences thereof with an identity of at least 95%, preferably 98% or more, to the corresponding nucleic acid and/or protein sequences and sequences coded by SEQ ID No. 1-491, partial sequences thereof and homologues thereof.

17. An arrangement of marker sequences for breast cancer, comprising one or more marker sequences according to claim 16.

18. A protein array comprising one or more marker sequences according to claim 16.

19. A diagnostic tool comprising one or more marker sequences according to claim 16 and optionally further additives and/or excipients.

20. A test kit comprising one or more marker sequences according to claim 16 and optionally further additives and/or excipients.

21. The arrangement according to claim 17, characterised in that 2 or 3, preferably 4 or 5, particularly preferably 7 or 8 or more, different marker sequences for breast cancer are used simultaneously.

22. Use of one or more marker sequences according to claim 16 for the early detection, diagnosis, prognosis, therapy control and/or aftercare in the case of breast cancer.

23. Use of one or more marker sequences according to claim 16 to distinguish breast cancer from benign changes.

24. Use of one or more marker sequences according to claim 16 for the individualized diagnosis and/or therapy in individual patients, patient groups, cohorts, population groups, variants of breast cancer, or stages of breast cancer.

25. Use of one or more marker sequences according to claim 16 for the detection and/or for the determination of the quantity of one or more breast cancer-associated autoantibodies, for example in bodily fluid or tissue of a patient.

26. Use of one or more marker sequences according to claim 16 for the analysis of autoantibody profiles of patients, in particular for the qualitative and/or quantitative analysis of autoantibodies and/or for the monitoring of changes of autoantibody profiles, for example in bodily fluids such as serum, tissue or tissue samples from the patient.

27. Use of one or more marker sequences according to claim 16 for the screening of substances (active agents) for breast cancer.

28. A target for the treatment and/or therapy of breast cancer selected from the marker sequences according to claim 16.

29. A method for the early detection, diagnosis, prognosis, therapy control and/or aftercare in the case of breast cancer, wherein
a.) a marker sequence or a number of marker sequences selected from the group comprising sequences SEQ ID No. 1-1473 and partial sequences of SEQ ID No. 1-1473 with at least 90%, preferably 95%, of the length of the sequences SEQ ID No. 1-1473 and homologues of SEQ ID No. 1-1473 and partial sequences thereof with an identity of at least 95%, preferably 98% or more, to the corresponding nucleic acid and/or protein sequences and sequences coded by SEQ ID No. 1-491, partial sequences thereof and homologues thereof is/are applied to a support,
b.) is/are brought into contact with bodily fluid or tissue sample from a patient, and
c.) an interaction of the bodily fluid or the tissue sample with the marker sequence(s) for breast cancer a from a.) is detected.
